(19) United States
(12) Patent Application Publication
(10) Pub. No.: US 2024/0366604 A1
(43) Pub. Date: Nov. 7, 2024

(54) SELECTIVE INHIBITORS OF ROCK2 FOR THE TREATMENT OF MUSCULAR DYSTROPHY

(71) Applicant: GRAVITON BIOSCIENCE BV, AMSTERDAM (NL)

(72) Inventor: Samuel D. Waksal, New York, NY (US)

(21) Appl. No.: 18/557,815

(22) PCT Filed: Apr. 28, 2022
(86) PCT No.: PCT/EP2022/061408

§ 371 Date: Oct. 27, 2023

Related U.S. Application Data
(60) Provisional application No. 63/181,121, filed on Apr. 28, 2021.

(51) Int. Cl. A61K 31/517; A61K 31/506; A61K 31/573; A61K 31/58; A61K 38/17; A61K 45/06; A61P 21/00; C12N 15/113

(52) U.S. Cl. CPC A61K 31/517; A61K 31/506; A61K 31/573; A61K 31/58; A61K 38/1719; A61K 45/06; A61P 21/00; C12N 15/1137; C12N 2310/11; C12N 2310/14; C12N 2310/141

(57) ABSTRACT

The disclosure provides compositions and methods comprising selective inhibitors of Rho-associated coiled-coil kinase 2 (ROCK2) for use in the treatment of muscular dystrophy, including Duchenne muscular dystrophy, Becker muscular dystrophy, facioscapulohumeral muscular dystrophy, limb-girdle muscular dystrophy, myotonic muscular dystrophy, oculopharyngeal muscular dystrophy, tibial muscular dystrophy, congenital muscular dystrophy, distal muscular dystrophy, and Emery-Dreifuss muscular dystrophy.

### CROSS-REFERENCE TO RELATED APPLICATIONS

This application claims priority to U.S. Provisional Application No. 63/181,121, filed Apr. 28, 2021, the contents of which are hereby incorporated by reference in their entirety.

### FIELD

The present disclosure relates to methods and compositions for the treatment of muscular dystrophy using selective inhibitors of Rho-associated coiled-coil kinase 2 (ROCK2).

### BACKGROUND

Muscular dystrophy is a group of inherited diseases that damage and weaken muscles over time, resulting from reduced or a lack function of the dystrophin protein, which is necessary for normal muscle function. The weakened muscles may result in problems, such as difficulty walking, swallowing, and muscle coordination. As cardiac and respiratory muscles weaken, patients may die from muscular dystrophy. Indeed, patients suffering from the most severe form of muscular dystrophy, Duchenne muscular dystrophy, often do not survive beyond their teens.

While, muscular dystrophy can occur at any age, most patients are diagnosed during childhood. Because the dystrophin gene is on the X chromosome, males are more likely to suffer from muscular dystrophy than females.

Dystrophic muscle comprises a fragile muscle membrane that may be damaged during muscle contraction, resulting in the replacement of muscle fibers with fibrous tissue over time. Because the fibrous tissue has no contractile properties, this replacement results in permanent weakness and disability. A muscular dystrophy patient's prognosis depends on the type of muscular dystrophy and the severity of symptoms, but most eventually lose the ability to walk and require a wheelchair. While certain treatments may help, there is currently no known cure for muscular dystrophy. Accordingly, there remains a need for additional treatments for muscular dystrophy.

Rho-associated coiled-coil kinase (ROCK) is a serine/threonine kinase from the AGC (PKA, PKG, and PKC) kinase family and comprises two isoforms: ROCK1 and ROCK2. The two isoforms are expressed and regulated differently in specific tissues. For example, ROCK1 is ubiquitously expressed at a relatively high level, while ROCK2 is preferentially expressed in certain tissues including heart, brain and skeletal muscle. ROCK is a target of the small GTPase Rho and is involved in diverse cellular activities achieved by phosphorylating downstream effector proteins (MLC, LIMK, ERM, MARCKS, CRMP-2, etc.). Studies have shown that various diseases (e.g., pulmonary fibrosis, cardiac-cerebral vascular disease, neurological disease and cancer etc.) are related to the pathways mediated by ROCK. As such, ROCK has been considered as an important target in the development of novel drugs.

### SUMMARY

The present disclosure relates to the previous unrecognized and surprising potent selective inhibitors of ROCK2 in use for the treatment of muscular dystrophy, including Duchenne muscular dystrophy, Becker muscular dystrophy, facioscapulohumeral muscular dystrophy, limb-girdle muscular dystrophy, myotonic muscular dystrophy, oculopharyngeal muscular dystrophy, tibial muscular dystrophy, congenital muscular dystrophy, distal muscular dystrophy, and Emery-Dreifuss muscular dystrophy.

In one aspect, the disclosure provides methods of treating muscular dystrophy in a subject in need thereof. In some embodiments, the method comprises administering to the subject a therapeutically effective amount of comprising a selective inhibitor of Rho-associated coiled-coil kinase 2 (ROCK2), or a composition comprising a selective inhibitor of ROCK2. In some embodiments, the muscular dystrophy is selected from the group consisting of Duchenne muscular dystrophy, Becker muscular dystrophy, facioscapulohumeral muscular dystrophy, limb-girdle muscular dystrophy, myotonic muscular dystrophy, oculopharyngeal muscular dystrophy, tibial muscular dystrophy, congenital muscular dystrophy, distal muscular dystrophy, and Emery-Dreifuss muscular dystrophy. In some embodiments, the muscular dystrophy is Duchenne muscular dystrophy.

In some embodiments, the selective inhibitor of ROCK2 is a compound according to Formula I, as defined herein, or a pharmaceutically acceptable salt, ester, stereoisomer, polymorph, solvate, N-oxide, or isotopically labeled compound thereof. In some embodiments, the selective inhibitor of ROCK2 is a compound according to any one of Formula II to IX, as defined herein, or a pharmaceutically acceptable salt, ester, stereoisomer, polymorph, solvate, N-oxide, or isotopically labeled compound thereof. In some embodiments, the selective inhibitor of ROCK2 is a compound according to Formula X or XI, as defined herein, or a pharmaceutically acceptable salt, ester, stereoisomer, polymorph, solvate, N-oxide, or isotopically labeled compound thereof. In some embodiments, the selective inhibitor of ROCK2 is a compound according to Formula XII, as defined herein, or a pharmaceutically acceptable salt, ester, stereoisomer, polymorph, solvate, N-oxide, or isotopically labeled compound thereof, and particularly (6-(4-((4-(1H-pyrazol-4-yl)phenyl)amino)pyrimidin-2-yl)-1-methyl-1H-indol-2-yl)(3,3-difluoroazetidin-1-yl)methanone.

In some embodiments, the selective inhibitor of ROCK2 is a compound according to Formula XIII, as defined herein, or a pharmaceutically acceptable salt, ester, stereoisomer, polymorph, solvate, N-oxide, or isotopically labeled compound thereof. In some embodiments, the selective inhibitor of ROCK2 is a compound according to Formula XIV, as defined herein, or a pharmaceutically acceptable salt, ester, stereoisomer, polymorph, solvate, N-oxide, or isotopically labeled compound thereof. In some embodiments, the selective inhibitor of ROCK2 is a compound according to Formula XV, as defined herein, or a pharmaceutically acceptable salt, ester, stereoisomer, polymorph, solvate, N-oxide, or isotopically labeled compound thereof. In some embodiments, the selective inhibitor of ROCK2 is a compound according to Formula XVI, as defined herein, or a pharmaceutically acceptable salt, ester, stereoisomer, polymorph, solvate, N-oxide, or isotopically labeled compound thereof. In some embodiments, the selective inhibitor of ROCK2 is a compound according to Formula XVII, as defined herein, or a pharmaceutically acceptable salt, ester, stereoisomer, polymorph, solvate, N-oxide, or isotopically labeled compound thereof, and particularly (2-(3-(4-((1H-indazol-5-yl)amino)quinazolin-2-yl)phenoxy)-N-isopropylacetamide).

In some embodiments, the selective inhibitor of ROCK2 is a ribonucleic acid (RNA). The RNA may be an antisense RNA against ROCK2 transcription. In some embodiments, the RNA is a small interfering RNA (siRNA) or micro RNA (miRNA).

In some embodiments, the subject is a human. In some embodiments, the subject has previous been administered an additional muscular dystrophy treatment. In some embodiments, the subject is concurrently being administered an additional muscular dystrophy treatment. In some embodiments, the subject will subsequently be administered an additional muscular dystrophy treatment.

In some embodiments, the additional muscular dystrophy treatment is selected from the group consisting of a corticosteroid, an angiotensin-converting enzyme (ACE) inhibitor, a beta blocker, an exon skipping oligonucleotide, and a gene therapy. In some embodiments, the corticosteroid is prednisone or deflazacort. In some embodiments, the exon skipping oligonucleotide is eteplirsen or golodirsen. In some embodiments, the gene therapy is SRP-9001 or PF-06939926.

### DETAILED DESCRIPTION

The compounds, compositions and methods described herein provide selective inhibitors of Rho-associated coiled-coil kinase 2 (ROCK2) for use in the treatment of muscular dystrophy, including Duchenne muscular dystrophy, Becker muscular dystrophy, facioscapulohumeral muscular dystrophy, limb-girdle muscular dystrophy, myotonic muscular dystrophy, oculopharyngeal muscular dystrophy, tibial muscular dystrophy, congenital muscular dystrophy, distal muscular dystrophy, and Emery-Dreifuss muscular dystrophy, particularly Duchenne muscular dystrophy.

Unless otherwise defined herein, scientific and technical terms used in this application shall have the meanings that are commonly understood by those of ordinary skill in the art. Generally, nomenclature used in connection with, and techniques of, organic chemistry, cell and tissue culture, molecular biology, immunology, microbiology, genetics and protein and nucleic acid chemistry and hybridization described herein are those well-known and commonly used in the art. In case of conflict, the present specification, including definitions, will control.

The methods and techniques of the present disclosure will employ, unless otherwise indicated, conventional techniques of organic chemistry, molecular biology (including recombinant techniques), microbiology, cell biology, biochemistry and immunology, which are within the skill of the art. Such techniques are explained fully in the literature, such as Green M R & Sambrook J. Molecular Cloning: A Laboratory Manual, 4th ed., Cold Spring Harbor Laboratory Press, Cold Spring Harbor, N.Y. (2012); Ausubel et al., Short Protocols in Molecular Biology: A Compendium of Methods from Current Protocols in Molecular Biology, 5th ed., Wiley, John & Sons, Inc. (2002); Harlow and Lane Using Antibodies: A Laboratory Manual, Cold Spring Harbor Laboratory Press, Cold Spring Harbor, N.Y. (1998); and Coligan et al., Short Protocols in Protein Science, Wiley, John & Sons, Inc. (2003); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Gene Transfer Vectors for Mammalian Cells (J. M. Miller and M. P. Calos, eds., 1987); Current Protocols in Molecular Biology (F. M. Ausubel et al., eds., John Wiley & Sons, Inc., 2003); PCR: The Polymerase Chain Reaction, (Mullis et al., eds., 1994), each of which is incorporated herein by reference.

Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those well-known and commonly used in the art.

Standard techniques are used for chemical syntheses, and chemical analyses.

Throughout this specification and embodiments, the word “comprise,” or variations such as “comprises” or “comprising,” will be understood to imply the inclusion of a stated integer or group of integers but not the exclusion of any other integer or group of integers. “Comprising” may be synonymous with “including” or “containing.”

It is understood that wherever embodiments are described herein with the language “comprising,” otherwise analogous embodiments described in terms of “consisting of” and/or “consisting essentially of” are also provided. As used herein, “consisting of” is a closed term that includes only the specific elements recited, and “consisting essentially of” includes the specific elements recited and may include additional unrecited, nonmaterial elements.

The term “including” is used to mean “including but not limited to.” “Including” and “including but not limited to” are used interchangeably.

Any example(s) following the term “e.g.” or “for example” is not meant to be exhaustive or limiting.

Unless otherwise required by context, singular terms shall include pluralities and plural terms shall include the singular.

As used herein, unless specifically indicated otherwise, the word “or” is used in the inclusive sense of “and/or” and not the exclusive sense of “either/or.”

As used herein, the term “and/or” is to be taken as specific disclosure of each of the two specified features or components with or without the other. For example, “A and/or B” is to be taken as specific disclosure of each of (i) A, (ii) B and (iii) A and B, just as if each were set out individually herein.

The articles “a”, “an” and “the” are used herein to refer to one or to more than one (i.e., to at least one) of the grammatical object of the article. By way of example, “an element” means one element or more than one element.

As used herein, the term “about” modifying the quantity of an ingredient, parameter, calculation, or measurement in the compositions of the disclosure or employed in the methods of the disclosure refers to variation in the numerical quantity that can occur, for example, through typical measuring and liquid handling procedures used for making isolated polypeptides or pharmaceutical compositions in the real world; through inadvertent error in these procedures; through differences in the manufacture, source, or purity of the ingredients employed to make the compositions or carry out the methods; and the like without having a substantial effect on the chemical or physical attributes of the compositions or methods of the disclosure. Such variation can be within an order of magnitude, typically within 10%, more typically still within 5%, of a given value or range. The term “about” also encompasses amounts that differ due to different equilibrium conditions for a composition resulting from a particular initial mixture. Whether or not modified by the term “about”, the paragraphs include equivalents to the quantities. Reference to “about” a value or parameter herein also includes (and describes) embodiments that are directed to that value or parameter per se. For example, description referring to “about X” also includes description of “X.” Numeric ranges are inclusive of the numbers defining the range.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10.

Exemplary methods and materials are described herein, although methods and materials similar or equivalent to those described herein can also be used in the practice or testing of the present application. The materials, methods, and examples are illustrative only and not intended to be limiting.

### Definitions

As used herein, the term “administering” or “administration” of a selective inhibitor of ROCK2 to a subject refers to contacting the selective inhibitor of ROCK2 to the subject or to a cell, tissue, organ, or biological fluid of the subject. Such administration includes any route of introducing or delivering the selective inhibitor of ROCK2 to perform the intended function. Administration can be carried out by any route suitable for the delivery of the selective inhibitor of ROCK2. Such administration can be carried out using one of a variety of methods known to those skilled in the art. For example, a selective inhibitor of ROCK2 of this disclosure can be administered systemically or locally. Thus, delivery routes can include oral, inhalational, transdermal, intravenous, intramuscular, intraperitoneal, or subcutaneous delivery. Administration includes self-administration and the administration by another. Administering can also be performed, for example, once, a plurality of times, and/or over one or more extended periods. In some embodiments, the administration includes both direct administration (including self-administration) and indirect administration, including the act of prescribing a drug.

As used herein, the term “composition” or “pharmaceutical composition” refers to a composition comprising a therapeutically effective compound and a pharmaceutically acceptable carrier. Optionally, the composition comprises other materials, e.g., one or more inert components (for example, a detectable agent or label) or one or more active components. The pharmaceutical composition facilitates administration of the therapeutically effective compound to a subject.

As used herein, the terms “carrier” and “pharmaceutically acceptable carrier” refer to a diluent, adjuvant, excipient, or vehicle in which the therapeutically effective compound is formulated. Pharmaceutically acceptable carriers may include one or more solvents, dispersion media, coatings, isotonic and absorption delaying agents, and the like that are physiologically compatible. Pharmaceutically acceptable carriers are well known in the art. See, e.g., Remington's Pharmaceutical Sciences and U.S. Pharmacopeia: National Formulary, Mack Publishing Company, Easton, PA (1984), incorporated herein by reference. Compositions can include components such as diluents, binders, stabilizers, buffers, salts, lipophilic solvents, preservatives, or mixtures thereof. Examples of pharmaceutically acceptable carriers include but are not limited to water, saline, phosphate buffered saline, aqueous dextrose solutions, glycerol solutions. Suitable pharmaceutical excipients include starch, glucose, lactose, sucrose, dextrose, gelatin, mannitol, cellulose malt, rice, flour, chalk, silica gel, sodium stearate, glycerol monostearate, talc, sodium chloride, milk powder, glycerol, propylene, glycol, water, ethanol and the like. Pharmaceutical compositions may be prepared by mixing a selective inhibitor of ROCK2 disclosed herein with acceptable carriers, excipients, or stabilizers in the form of, e.g., lyophilized powders, slurries, aqueous solutions or suspensions (see, e.g., Hardman, et al. (2001) Goodman and Gilman's The Pharmacological Basis of Therapeutics, McGraw-Hill, New York, NY; Gennaro (2000) Remington: The Science and Practice of Pharmacy, Lippincott, Williams, and Wilkins, New York, NY; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, NY; each incorporated herein by reference).

As used herein, the terms “patient” “subject” “individual” and the like are used interchangeably herein, and refer to any animal, or cells thereof whether in vitro or in situ, amenable to the methods described herein. In certain non-limiting embodiments, the patient, subject or individual is a human.

As used herein, the terms “polynucleotide” “nucleic acid” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double and single stranded molecules. Unless otherwise specified or required, any embodiment of this disclosure that is a polynucleotide encompasses both the double stranded form and each of two complementary single stranded forms known or predicted to make up the double stranded form.

As used herein, the terms “therapeutically effective amount” and “effective amount” with regard to a compound or a composition refer to an amount sufficient to confer a therapeutic benefit in a patient after administration, for example, to improve in the subject one or more symptoms of the disease, or to delay, reduce, minimize, mitigate, or ameliorate the symptoms of a disease relative to an untreated patient. The effective amount may vary depending on the species, age, weight, health of the subject and the nature or severity of the disease. Depending on the mode of administration, the effective amount may vary as well. In some cases, multiple doses of the composition are administered to achieve the effective amount for the therapeutic benefit intended. A therapeutically effective amount is also one in which any toxic or detrimental effects of the selective inhibitor of ROCK2 are outweighed by the therapeutically beneficial effects. It is routine in the art for the skilled artisan to determine a therapeutically effective amount of a selective inhibitor of ROCK2 disclosed herein based on these factors. A “prophylactically effective amount” refers to an amount effective, at dosages and for periods of time necessary, to achieve the desired prophylactic result. Typically, since a prophylactic dose is used in subjects prior to transplantation or at an earlier stage of transplant rejection, the prophylactically effective amount may be less than the therapeutically effective amount.

As used herein, the terms “treat,” “treating,” and “treatment” refer the administration of a therapeutic agent, such as a selective inhibitor of ROCK2 or a composition containing any such selective inhibitor of ROCK2 disclosed herein, internally or externally to a subject or patient having one or more disease symptoms, or being suspected of having a muscular dystrophy. “Treat,” “treating” and “treatment” refer to therapeutic treatments and/or prophylactic treatments. These terms cover the treatment of a muscular dystrophy, in a subject, such as a human, and include: (i) inhibiting muscular dystrophy, i.e., arresting its development; (ii) relieving muscular dystrophy, i.e., causing regression of the disorder; (iii) slowing progression of muscular dystrophy; and/or (iv) inhibiting, relieving, or slowing progression of one or more symptoms of muscular dystrophy. Treatment and treating refers to the intentional act of physiological intervention that is intended to cure, retard, or ameliorate one or more symptoms associated with muscular dystrophy. If the treatment is administered prior to clinical manifestation of muscular dystrophy, the treatment is considered prophylactic. The alleviation or reduction of a symptom of muscular dystrophy can be assessed by any clinical measurement typically used by physicians or other skilled artisans to assess the severity or progression of that symptom. The terms further refer to a postponement of development of one or more muscular dystrophy symptoms and/or a reduction in the severity of one or more muscular dystrophy symptoms. The terms further include ameliorating existing uncontrolled or unwanted muscular dystrophy symptoms, preventing additional muscular dystrophy symptoms, and ameliorating or preventing the underlying causes of such symptoms. Thus, the terms denote that a beneficial result has been conferred on the subject.

As used herein, the terms “prevent” and “prevention” refer to acting prophylactically prior to overt muscular dystrophy onset, to prevent it from developing, or to minimize the extent of it, or to slow its course of development.

As used herein, the term “stability” can refer to chemical stability and/or physical stability. As used herein, the term “chemical stability” can refer to the ability of a compound to maintain its chemical identity over time. Accordingly, stability implies the ability of a chemical species to resist oxidation or other degradations. As used herein, the phrase “physical stability” can refer to the ability of a composition to maintain consistent physical properties over time. The ability of a composition to maintain a consistent disintegration time over time is exemplary of physical stability.

### Selective Inhibitors of ROCK2

The compounds for use in the methods and compositions disclosed herein selective inhibitors of ROCK2. The compounds provide excellent inhibitory activity of ROCK2, higher selectivity towards ROCK2 as compared with ROCK1, good physicochemical properties (e.g., solubility, physical and/or chemical stability), improved pharmacokinetic properties (e.g., improved bioavailability, proper half-life and duration of action), improved safety (low toxicity and/or less side effects, wide therapeutic window), and the like. In some embodiments, the selective inhibitor of ROCK2 is at least 10-fold more selective for ROCK2 than Rho associated kinase 1 (ROCK1). In some embodiments, the selective inhibitor of ROCK2 is at least 20-fold, 30-fold, 40-fold, 50-fold, 60-fold, 70-fold, 80-fold, 90-fold, or 100-fold more selective for ROCK2 than ROCK1.

As provided herein, selective inhibitor of ROCK2 have previously unrecognized and surprisingly potent effects in the treatment of muscular dystrophy, including Duchenne muscular dystrophy, Becker muscular dystrophy, facioscapulohumeral muscular dystrophy, limb-girdle muscular dystrophy, myotonic muscular dystrophy, oculopharyngeal muscular dystrophy, tibial muscular dystrophy, congenital muscular dystrophy, distal muscular dystrophy, and Emery-Dreifuss muscular dystrophy, particularly Duchenne muscular dystrophy. Without being bound by theory, selective inhibitors of ROCK2, such as, for example, compounds of the Formulae I to XVII, may interfere with the replacement of damaged muscle tissues with non-contractile fibrous tissue.

According to one aspect, the present disclosure provides methods of treating a subject by administering to the subject a therapeutically effective amount of a selective inhibitor of ROCK2, or a composition comprising the selective inhibitor of ROCK2. Any selective inhibitor of ROCK2 disclosed herein may be used in the methods disclosed herein. Non-limiting examples of selective inhibitors of ROCK2 include the selective inhibitors of ROCK2 disclosed in U.S. 2019/0276440, the entire disclosure of which is hereby incorporated by reference herein. In some embodiments, the selective inhibitor of ROCK2 is a compound according to Formula I:

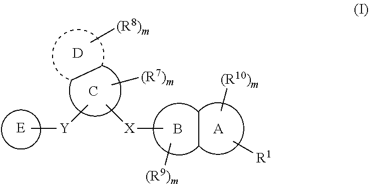

or a pharmaceutically acceptable salt, ester, stereoisomer, polymorph, solvate, N-oxide, or isotopically labeled compound thereof, wherein:
X and Y are each independently selected from the group consisting of a direct bond, C(═O), O, S(═O)ᵢ and NR;
R is selected from the group consisting of H, C₁₋₆ alkyl, C₂₋₆ alkenyl, C₂₋₆ alkynyl, saturated or partially unsaturated C₃₋₁₀ cyclic hydrocarbyl, saturated or partially unsaturated 3- to 10-membered heterocyclyl, C₆₋₁₀ aryl, 5- to 14-membered heteroaryl and C₆₋₁₂ aralkyl, and at most 2 ring members in the cyclic hydrocarbyl and heterocyclyl are C(═O);
ring A and ring B are each independently selected from the group consisting of saturated or partially unsaturated C₃₋₁₀ hydrocarbon ring, saturated or partially unsaturated 3- to 10-membered heterocycle, C₆₋₁₀ aromatic ring and 5- to 14-membered heteroaromatic ring, and at most 2 ring members in the hydrocarbon ring and heterocycle are C(═O); provided that when ring B is a heterocycle containing a nitrogen atom, ring B is not attached to X via the nitrogen atom;
ring C is selected from the group consisting of saturated or partially unsaturated C₃₋₁₀ hydrocarbon ring, saturated or partially unsaturated 3- to 10-membered heterocycle, C₆₋₁₀ aromatic ring and 5- to 14-membered heteroaromatic ring, and at most 2 ring members in the hydrocarbon ring and heterocycle are C(═O);
ring D is absent, or is selected from the group consisting of saturated or partially unsaturated C₃₋₁₀ hydrocarbon ring, saturated or partially unsaturated 3- to 10-membered heterocycle, C₆₋₁₀ aromatic ring and 5- to 14-membered heteroaromatic ring, and at most 2 ring members in the hydrocarbon ring and heterocycle are C(═O)
ring E is selected from the group consisting of;

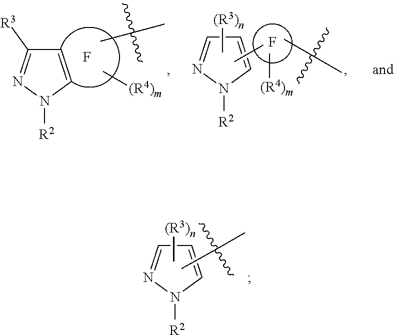

ring F is selected from the group consisting of saturated or partially unsaturated C₃₋₁₀ hydrocarbon ring, saturated or partially unsaturated 3- to 10-membered heterocycle, C₆₋₁₀ aromatic ring and 5- to 14-membered heteroaromatic ring, and at most 2 ring members in the hydrocarbon ring and heterocycle are C(═O);
R¹ is selected from the group consisting of H, —NH₂, C₁₋₆ alkyl, C₆₋₁₀ aryl, 5- to 14-membered heteroaryl, N-methylpyrrolidinyl, N-methylpiperidinyl,

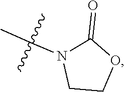 [SMILES: *N1CCOC1=O]

acetyl,

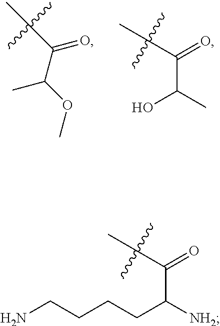

—C(═O)—(C₁₋₆ alkylene)n-CF₃, —C(═O)—(C₁₋₆ alkylene) CN, —C(═O)-(saturated or partially unsaturated C₃₋₁₀ cyclic hydrocarbyl), —NHC(═O)-(saturated or partially unsaturated C₃₋₁₀ cyclic hydrocarbyl), —C(═O)-(saturated or partially unsaturated 3- to 10-membered heterocyclyl), —C(═O)—C₁₋₆ alkylene-(saturated or partially unsaturated 3- to 10-membered heterocyclyl), —C(═O)-(5- to 14-membered heteroaryl), —C(═O)—C₁₋₆ alkylene-NH(C₁₋₆ alkyl), —C(═O)—C₁₋₆ alkylene-N(C₁₋₆ alkyl)₂, N-methylpiperazine substituted acetyl, —S(═O)₂R^{1a}, —P(═O)R^{1a}R^{1b},

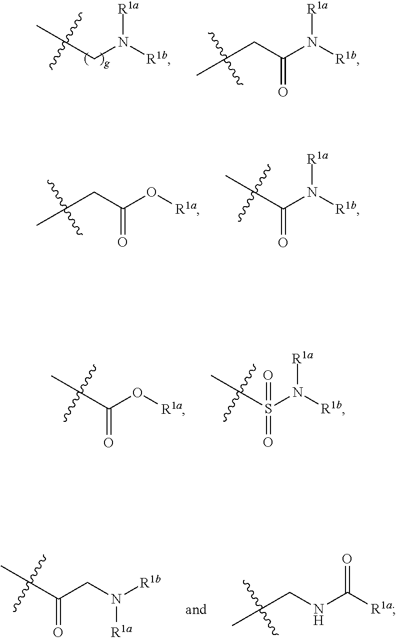

provided that when one of R¹ and R¹⁰ is C¹⁻⁶ alkyl, and the other is H or C³⁻¹⁰ cyclic hydrocarbyl, at least one of X and Y is a direct bond, and ring C is not a 5-membered heteroaromatic ring; when one of R¹ and R¹⁰ is H, and the other is

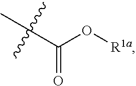

ring C is not a 5-membered heteroaromatic ring; when both R¹ and R¹⁰ are H, ring A contains at least one nitrogen atom, and is not a 5- or 6-membered ring; when one of R¹ and R¹⁰ is H, and the other is

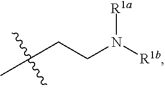

ring C is not a 5-membered heteroaromatic ring; and when one of R¹ and R¹⁰ is H, and the other is H or acetyl, ring D is absent;
R^{1a} and R^{1b} are each independently selected from the group consisting of H, halogen, amino, cyano, nitro, C₁₋₆ alkyl, C₂₋₆ alkenyl, C₂₋₆ alkynyl, C₃₋₁₀ cyclic hydrocarbyl, 3- to 10-membered heterocyclyl, C₆₋₁₀ aryl, 5- to 14-membered heteroaryl, C₆₋₁₂ aralkyl,
—C(═O)R⁵, —OC(═O)R⁵, —C(═O)OR⁵, —OR⁵, —SR⁵, —S(═O)R⁵, —S(═O)₂R⁵,
—S(═O)₂NR⁵R⁶, —NR⁵R⁶, —C(═O)NR⁵R⁶, —NR⁵—C(═O)R⁶, —NR⁵—C(═O)OR⁶,
NR⁵—S(═O)₂—R⁶, —NR⁵—C(═O)—NR⁵R⁶, —C₁₋₆ alkylene-NR⁵R⁶, —C₁₋₆ alkylene-OR⁵ and
—O—C₁₋₆ alkylene-NR⁵R⁶, provided that when one of R^{1a} and R^{1b} is n-propyl, the other is not H; or R^{1a} and R^{1b} together with the atom to which they are attached form a 3- to 12-membered heterocycle or heteroaromatic ring;
R², R³, R⁴, R⁷, R⁸, R⁹ and R¹⁰, at each occurrence, are each independently selected from the group consisting of H, halogen, amino, cyano, nitro, C₁₋₆ alkyl, C₂₋₆ alkenyl, C₂₋₆ alkynyl, C₃₋₁₀ cyclic hydrocarbyl, 3- to 10-membered heterocyclyl, C₆₋₁₀ aryl, 5- to 14-membered heteroaryl, C₆₋₁₂ aralkyl, —C(═O)R⁵, OC(═O)R⁵, —C(═O)OR⁵, OR⁵, SR⁵, —S(═O)R⁵, —S(═O)₂R⁵, —S(═O)₂NR⁵R⁶, —NR⁵R⁶, —C(═O)NR⁵R⁶, —NR⁵—C(═O)R⁶,
—NR⁵—C(═O)OR⁶, —NR⁵—S(═O)₂—R⁶, —NR⁵—C(═O)—NR⁵R⁶, —C₁₋₆ alkylene-NR⁵R⁶,
—C₁₋₆ alkylene-O(P═O)(OH)₂ and —O—C₁₋₆ alkylene-NR⁵R⁶;
the above alkyl, alkylene, alkenyl, alkynyl, cyclic hydrocarbyl, hydrocarbon ring, heterocyclyl, heterocycle, aryl, aromatic ring, heteroaryl, heteroaromatic ring and aralkyl, at each occurrence, are each optionally substituted with one or more substituents independently selected from the group consisting of halogen, hydroxyl, oxo, amino, cyano, nitro, C₁₋₆ alkyl, C₂₋₆ alkenyl, C₂₋₆ alkynyl, C₃₋₆ cyclic hydrocarbyl, 3- to 10-membered heterocyclyl, C₆₋₁₀ aryl, 5- to 14-membered heteroaryl, C₆₋₁₂ aralkyl, ═N—OR⁵, —C(═NH)NH₂, —C(═O)R⁵, —OC(═O)R⁵, —C(═O)OR⁵, —OR⁵, —SR⁵, —S(═O)R⁵, —S(═O)₂R⁵, —S(═O)₂NR⁵R⁶, —NR⁵R⁶, —C(═O)NR⁵R⁶, —NR⁵—C(═O)R⁶, —NR⁵—C(═O)OR⁶, —NR⁵—S(═O)₂—R⁶, —NR⁵—C(═O)—NR⁵R⁶, —C₁₋₆ alkylene-NR⁵R⁶ and —O—C₁₋₆ alkylene-NR⁵R⁶, and the alkyl, cyclic hydrocarbyl, heterocyclyl, aryl, heteroaryl and aralkyl are further optionally substituted with one or more substituents independently selected from the group consisting of halogen, hydroxyl, oxo, amino, cyano, nitro, C₁₋₆ alkyl, C₃₋₆ cyclic hydrocarbyl, 3- to 10-membered heterocyclyl, C₆₋₁₀ aryl, 5- to 14-membered heteroaryl and C₆₋₁₂ aralkyl;
R⁵ and R⁶, at each occurrence, are each independently selected from the group consisting of H, alkyl, C₃₋₁₀ cyclic hydrocarbyl, 3- to 10-membered heterocyclyl, C₆₋₁₀ aryl, 5- to 14-membered heteroaryl and C₆₋₁₂ aralkyl;
m, at each occurrence, is each independently an integer of 0, 1, 2 or 3;
n is an integer of 0, 1 or 2;
i is an integer of 0, 1 or 2; and
g is an integer of 0, 1, 2, 3 or 4.

In some embodiments, the selective inhibitor of ROCK2 is a compound according to any one of Formula II to IX:

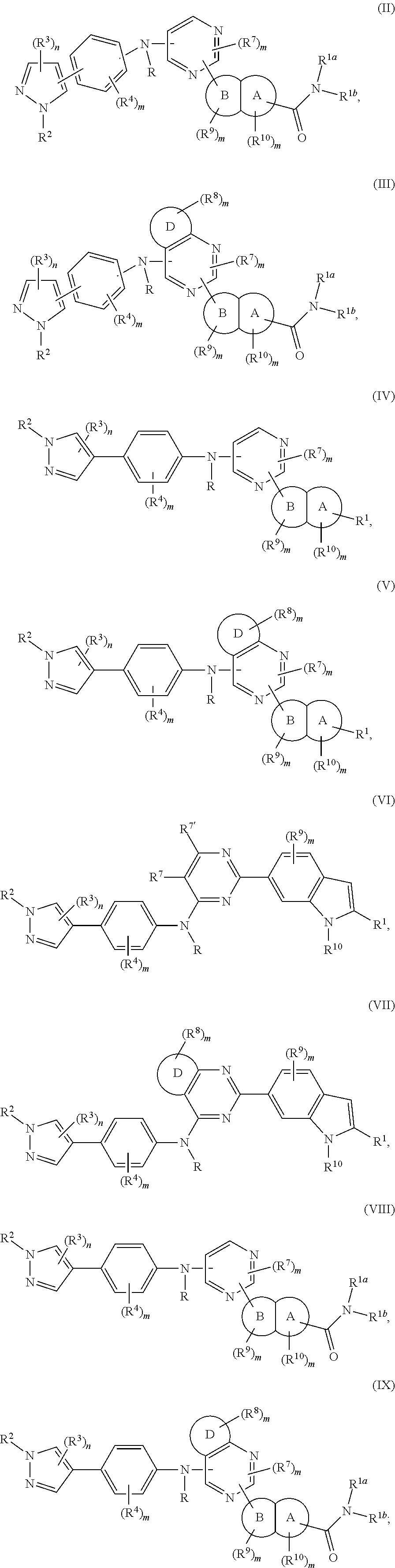

or a pharmaceutically acceptable salt, ester, stereoisomer, polymorph, solvate, N-oxide, or isotopically labeled compound thereof, wherein each of ring A, ring B, ring D, R, R¹, R^{1a}, R^{1b}, R², R³, R⁴, R⁷, R^{7′}, R⁸, R⁹, R¹⁰, n and m are defined above for Formula I.

In some embodiments, the selective inhibitor of ROCK2 is a compound according to Formula (X) or Formula (XI):

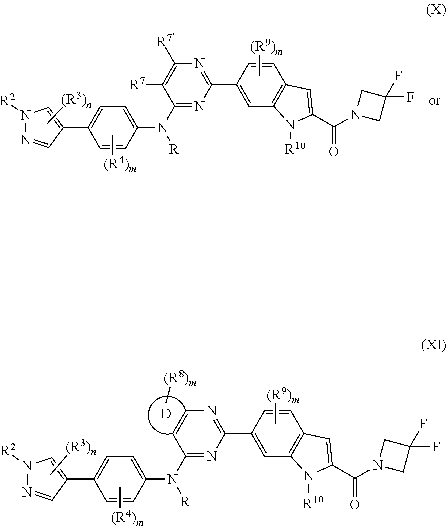

or a pharmaceutically acceptable salt, ester, stereoisomer, polymorph, solvate, N-oxide, or isotopically labeled compound thereof, wherein:
R is selected from the group consisting of H and C₁₋₆ alkyl;
ring D is saturated or partially unsaturated 3- to 10-membered heterocycle, C₆₋₁₀ aryl, or 5- to 10-membered heteroaromatic ring, preferably

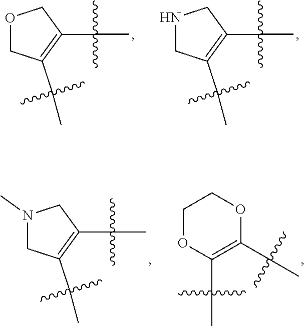

phenyl ring, N-methylpyrrole ring, furan ring or thiophene ring;
R² is selected from the group consisting of H and C₁₋₆ alkyl;
R³, R⁴, R⁷, R^{7′} and R⁸, at each occurrence, are each independently selected from the group consisting of H, halogen, —NH₂, —OH, C₁₋₆ alkyl and —OR⁵;
R⁹ and R¹⁰, at each occurrence, are each independently selected from the group consisting of H, halogen, C₁₋₆ alkyl, C₂₋₆ alkenyl, C₃₋₁₀ cyclic hydrocarbyl, 3- to 10-membered heterocyclyl, C₆₋₁₀ aryl, 5- to 14-membered heteroaryl, C₆₋₁₂ aralkyl, —C(═O)R⁵ and
—C₁₋₆ alkylene-O(P═O)(OH)₂;
the above alkyl, alkenyl, cyclic hydrocarbyl, heterocyclyl, aryl, heteroaryl, heteroaromatic ring and aralkyl, at each occurrence, are each optionally substituted with one or more substituents independently selected from the group consisting of halogen, C₁₋₆ alkyl and —OR⁵;
R⁵ and R⁶, at each occurrence, are each independently selected from the group consisting of H, C₁₋₆ alkyl, C₃₋₁₀ cyclic hydrocarbyl, 3- to 10-membered heterocyclyl, C₆₋₁₀ aryl, 5- to 14-membered heteroaryl and C₆₋₁₂ aralkyl;
m, at each occurrence, is each independently an integer of 0, 1, 2 or 3; and
n is an integer of 0, 1 or 2.

In preferred embodiments, R⁵ and R⁶, at each occurrence, are each independently selected from the group consisting of H, methyl and ethyl.

In preferred embodiments, R³, R⁴, R⁷, R^{7′} and R⁸, at each occurrence, are each independently selected from the group consisting of H, F, Cl, Br, —NH₂, —OH, methyl, trifluoromethyl, —CH₂-Ph, methoxy, ethoxy and —CH₂OCH₃.

In preferred embodiments, R⁹ and R¹⁰, at each occurrence, are each independently selected from the group consisting of H, F, Cl, Br, methyl, ethyl, n-propyl, isopropyl, vinyl, cyclopropyl, cyclobutyl, cyclopentyl, oxetanyl, monofluoromethyl, difluoromethyl, trifluoromethyl, acetyl, —OCH₂CHF₂, CH₂OH, —CH₂OCH₃, —CH₂CH₂OCH₃, —CH₂—O(P═O)(OH)₂,

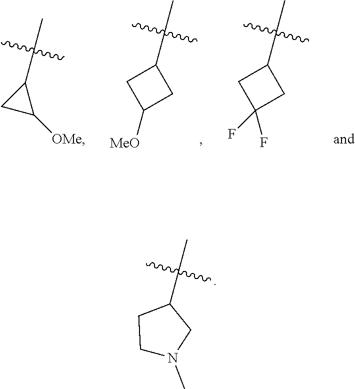

In some embodiments, the selective inhibitor of ROCK2 is a compound (6-(4-((4-(1H-pyrazol-4-yl)phenyl)amino)pyrimidin-2-yl)-1-methyl-1H-indol-2-yl)(3,3-difluoroazetidin-1-yl)methanone having the structure of Formula XII:

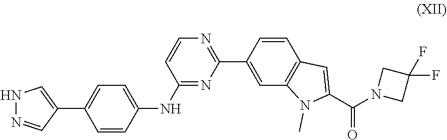 [SMILES: Cn1c(C(=O)N2CC(F)(F)C2)cc2ccc(-c3nccc(Nc4ccc(-c5cn[nH]c5)cc4)n3)cc21]

or a pharmaceutically acceptable salt, ester, stereoisomer, polymorph, solvate, N-oxide, or isotopically labeled compound thereof.

Compounds of the Formula I to XII, and particularly (6-(4-((4-(1H-pyrazol-4-yl)phenyl)amino)pyrimidin-2-yl)-1-methyl-1H-indol-2-yl)(3,3-difluoroazetidin-1-yl)methanone, may be prepared according to the methods disclosed in U.S. 2019/0276440, the contents of which is incorporated herein in its entirety.

Additional non-limiting examples of selective inhibitors of ROCK2 include the selective inhibitors of ROCK2 disclosed in WO 2014/055996, the entire disclosure of which is hereby incorporated by reference herein. In some embodiments, the selective inhibitor of ROCK2 is a compound according to Formula XIII:

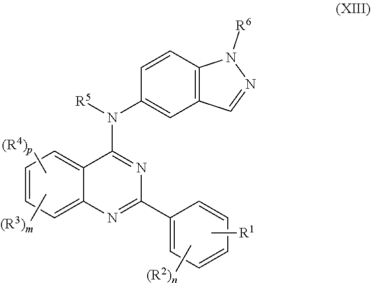

or a pharmaceutically acceptable salt, ester, stereoisomer, polymorph, solvate, N-oxide, or isotopically labeled compound thereof, wherein:
R¹ is selected from the group consisting of —O—(CH₂)ᵣCO₂R¹², —O—(CH₂)_{y}, —C(═O)NR¹³R¹⁴, —O—(CH₂)_{y}, -heteroaryl, —O—(CH₂)_{y}, -cycloalkyl, —O—C(═O)—(CH₂)_{y}, —NR¹³R¹⁴, —O—(CH₂)_{z}—NR¹³R¹⁴, —NH—C(═O)—(CH₂)_{y}, —NR¹³R¹⁴, —NH—C(═O)—X—R¹⁵, and —NH—(CH₂)_{y}, NR¹³R¹⁴;
R¹² is selected from the group consisting of C₁-C₆ alkyl, —(C₁-C₆ alkyl)-O—(C₁-C₆alkyl), —(C₁-C₆ alky)-NR¹⁶R¹⁷, —(C₁-C₆ alkyl)-C(═O)NR¹⁶R¹⁷, —(C₁-C₆ alkyl)-O—(C₁-C₆ alkyl)-O—(C₁-C₆ alkyl), aryl, aralkyl, heteroaryl, C₃-C₇ cycloalkyl, a three to twelve membered heterocyclic ring containing up to 3 heteroatoms, each of which may be optionally substituted at one or more carbon atoms by from 1 to 3 substituents independently selected from halo, C₁-C₆ alkoxy, hydroxy, amino, cyano and C₁-C₃ perfluoro alkyl;
R¹³ and R¹⁴ are independently selected from the group consisting of H, C₁-C₈ alkyl, C₂-C₈ alkenyl, C₂-C₈ alkynyl, —(C₁-C₆ alkyl)-O—(C₁-C₆ alkyl), —(C₁-C₆ alkyl)-NR¹⁶R¹⁷, —(C₁-C₆alkyl)-C(═O)NR¹⁶R¹⁷, aryl, aralkyl, heteroaryl, C₃-C₇ cycloalkyl, a three to twelve membered heterocyclic ring containing up to 3 heteroatoms, each of which may be optionally substituted by from 1 to 3 substituents independently selected from halo, C₁-C₆ alkyl, C₂-C₆ alkenyl, C₃-C₇ cycloalkyl, C₁-C₆ alkoxy, hydroxy, amino, cyano and C₁-C₃ perfluoro alkyl;
or R¹³ and R¹⁴ may be taken together to form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, C₃-C₇ cycloalkyl, oxo, hydroxy, amino, cyano and C₁-C₃ perfluoro alkyl;
X is absent or selected from a —O, NH, and C₁-C₆ alkyl;
R¹⁵ is selected from the group consisting of heteroaryl, C₃-C₇ cycloalkyl, a three to twelve membered heterocyclic ring containing up to 3 heteroatoms, each of which may be optionally substituted by from 1 to 3 substituents independently selected from halo, C₁-C₆ alkyl, C₂-C₆ alkenyl, C₁-C₆ alkoxy, hydroxy, amino, cyano and C₁-C₃ perfluoro alkyl,
or R¹⁵ is selected from —(C₁-C₆ alkyl)-O—(C₁-C₆ alkyl), —(C₁-C₆ alkyl)-NR¹⁶R¹⁷, —CO₂R¹⁸, —O—(CH₂)ₓ—CO₂R¹⁸, and —C(═O)NR¹⁶R¹⁷;
R¹⁶ and R¹⁷ are independently selected from the group consisting of H, C₁-C₈ alkyl, C₂-C₈ alkenyl, C₂-C₈ alkynyl, —(C₁-C₆ alkyl)-O—(C₁-C₆ alkyl), aryl, aralkyl, heteroaryl, C₃-C₇ cycloalkyl, a three to twelve membered heterocyclic ring containing up to 3 heteroatoms, each of which may be optionally substituted by from 1 to 3 substituents independently selected from halo, C₁-C₆ alkyl, C₂-C₆ alkenyl, C₁-C₆ alkoxy, hydroxy, amino, cyano and C₁-C₃ perfluoro alkyl;
or R¹⁶ and R¹⁷ may be taken together to form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆alkoxy, oxo, hydroxy, amino, cyano and C₁-C₃ perfluoro alkyl;
R¹⁸ is selected from the group consisting of H, aryl, aralkyl, heteroaryl, C₁-C₆ alkyl, (C₁-C₆alkyl)-O—(C₁-C₆ alkyl), —(C₁-C₆ alkyl)-NR¹⁶R¹⁷, —(C₁-C₆alkyl)-O—(C₁-C₆ alkyl)-O—(C₁-C₆ alkyl), each of which may be optionally substituted by from 1 to 3 substituents independently selected from halo, C₁-C₆ alkoxy, hydroxy, amino, cyano and C₁-C₃ perfluoroalkyl;
x is selected from 0 to 6;
y is selected from 0 to 6;
z is selected from 2 to 6;
each R² is independently selected from the group consisting of lower alkyl, CN, halo, hydroxy, lower alkoxy, amino, and perfluoro lower alkyl;
each R³ is independently selected from the group consisting of lower alkyl, CN, halo, hydroxy, lower alkoxy, amino, and perfluoro lower alkyl;
R⁴ is selected from H, —(CH₂)ₐ—NR⁴³R⁴⁴, —Y—R⁴², —O—(CH₂)ₐ—CO₂R⁴², —O—(CH₂)ₐ—C(═O)NR⁴³R⁴⁴, —O—(CH₂)ₐ-heteroaryl, —O—(CH₂)ₐ-cycloalkyl, —O—C(═O)—(CH₂)ₐ—NR⁴³R⁴⁴, —O—(CH₂)_{c}—N⁴³R⁴⁴, —NH—C(═O)—Y—R⁴⁵, —NH—C(═O)—(CH₂)ₐ—NR⁴³R⁴⁴;
R⁴² is selected from the group consisting of C₁-C₆ alkyl, —(C₁-C₆ alkyl)-O—(C₁-C₆ alkyl), —(C₁-C₆ alkyl)-NR⁴⁶R⁴⁷, (C₁-C₆ alkyl)-C(═O)N⁴⁶R⁴⁷, —(C₁-C₆ alkyl)-O—(C₁-C₆ alkyl)-O—(C₁-C₆ alkyl), each of which may be optionally substituted at one or more carbon atoms by from 1 to 3 substituents independently selected from halo, C₁-C₆alkoxy, hydroxy, amino, cyano and C₁-C₃ perfluoro alkyl;
R⁴³ and R⁴⁴ are independently selected from the group consisting of H, C₁-C₈ alkyl, C₂-C₈ alkenyl, C₂-C₈ alkynyl, —(C₁-C₆ alkyl)-O—(C₁-C₆ alkyl), —(C₁-C₆ alkyl)-NR⁴⁶R⁴⁷, —(C₁-C₆ alkyl)-C(═O)NR⁴⁶R⁴⁷, aryl, aralkyl, heteroaryl, C₃-C₇ cycloalkyl, a three to twelve membered heterocyclic ring containing up to 3 heteroatoms, each of which may be optionally substituted by from 1 to 3 substituents independently selected from halo. C₁-C₆ alkyl, C₂-C₆ alkenyl, C₃-C₇ cycloalkyl, C₁-C₆ alkoxy, hydroxy, amino, cyano and C₁-C₃ perfluoro alkyl;
or R⁴³ and R⁴⁴ may be taken together to form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆alkoxy, oxo, hydroxy, amino, cyano and C₁-C₃ perfluoro alkyl;
Y is absent or selected from O, NH, and C₁-C₆ alkyl;
R⁴⁵ is selected from the group consisting of H, aryl, —(C₁-C₆ alkyl)-O—(C₁-C₆ alkyl), —(C₁-C₆ alkyl)-NR⁴⁶R⁴⁷, —CO₂R⁴⁸, —O—(CH₂)₆—CO₂R⁴⁸, and —C(═O)NR⁴⁶R⁴⁷,
R⁴⁶ and R⁴⁷ independently selected from the group consisting of H, C₁-C₈ alkyl, C₂-C₈ alkenyl, C₂-C₈ alkynyl, —(C₁-C₆ alkyl)-O—(C₁-C₆ alkyl), aryl, aralkyl, heteroaryl, C₃-C₇ cycloalkyl, a three to twelve membered heterocyclic ring containing up to 3 heteroatoms, each of which may be optionally substituted by from 1 to 3 substituents independently selected from halo, C₁-C₆ alkyl, C₂-C₆ alkenyl, C₁-C₆ alkoxy, hydroxy, amino, cyano and C₁-C₃ perfluoro alkyl;
or R⁴⁶ and R⁴⁷ may be taken together to form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, oxo, hydroxy, amino, cyano and C₁-C₃ perfluoro alkyl;
R⁴⁸ is selected from the group consisting of H, aryl, aralkyl, heteroaryl, C₁-C₆ alkyl, —(C₁-C₆ alkyl)-O—(C₁-C₆ alkyl), —(C₁-C₆ alkyl)-NR⁴⁶R⁴⁷, —(C₁-C₆ alkyl)-O—(C₁-C₆alkyl)-O—(C₁-C₆ alkyl), each of which may be optionally substituted by from 1 to 3 substituents independently selected from halo, C₁-C₆ alkoxy, hydroxy, amino, cyano and C₁-C₃ perfluoroalkyl;
a is selected from 0 to 6;
b is selected from 0 to 6;
c is selected from 2 to 6;
R⁵ is selected from the group consisting of H, C₁-C₆ alkyl, (CH₂)ₐ—C(═)—NR⁵³R⁵⁴, —C(═O)—(CH₂)_{d}—NR⁵³R⁵⁴, and —C(═O)—X—R⁵⁵;
R⁵³ and R⁵⁴ are independently selected from the group consisting of H, C₁-C₈ alkyl, C₂-C₈ alkenyl, C₂-C₈ alkynyl, —(C₁-C₆ alkyl)-O—(C₁-C₆ alkyl), —(C₁-C₆ alkyl)-NR⁵⁶R⁵⁷, —(C₁-C₆ alkyl)-C(═O)NR⁵⁶R⁵⁷, aryl, aralkyl, heteroaryl, C₃-C₇ cycloalkyl, a three to twelve membered heterocyclic ring containing up to 3 heteroatoms, each of which may be optionally substituted by from 1 to 3 substituents independently selected from halo, C₁-C₆ alkyl, C₂-C₆ alkenyl, C₃-C₇ cycloalkyl, C₁-C₆ alkoxy, hydroxy, amino, cyano and C₁-C₃ perfluoro alkyl;
or R⁵³ and R⁵⁴ may be taken together to form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, C₃-C₇ cycloalkyl, oxo, hydroxy, amino, cyano and C₁-C₃ perfluoro alkyl;
R⁵⁵ is selected from the group consisting of H, aryl, —(C₁-C₆ alkyl)-O—(C₁-C₆ alkyl), (C₁-C₆alkyl)-NR⁵⁶R⁵⁷, —CO₂R⁵⁸, —O—(CH₂)ₑ—CO₂R⁵⁸, and —C(═O)NR⁵⁶R⁵⁷,
R⁵⁶ and R⁵⁷ independently selected from the group consisting of H, C₁-C₈ alkyl, C₂-C₈ alkenyl, C₂-C₈ alkynyl, (C₁-C₆ alkyl)-O—(C₁-C₆ alkyl), aryl, aralkyl, heteroaryl, C₃-C₇ cycloalkyl, a three to twelve membered heterocyclic ring containing up to 3 heteroatoms, each of which may be optionally substituted by from 1 to 3 substituents independently selected from halo, C₁-C₆ alkyl, C₂-C₆ alkenyl, C₁-C₆ alkoxy, hydroxy, amino, cyano and C₁-C₃ perfluoro alkyl;
or R⁵⁶ and R⁵⁷ may be taken together to form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, oxo, hydroxy, amino, cyano and C₁-C₃ perfluoro alkyl;
R⁵⁸ is selected from the group consisting of H, aryl, aralkyl, heteroaryl, C₁-C₆ alkyl, (C₁-C₆ alkyl)-O—(C₁-C₆ alkyl), —(C₁-C₆ alkyl)-NR⁵⁶R⁵⁷, —(C₁-C₆ alkyl)-O—(C₁-C₆alkyl)-O—(C₁-C₆ alkyl), each of which may be optionally substituted by from 1 to 3 substituents independently selected from halo, C₁-C₆ alkoxy, hydroxy, amino, cyano and C₁-C₃ perfluoroalkyl:
d is selected from 0 to 6;
e is selected from 0 to 6;
R⁶ is selected from the group consisting of H, C₁-C₆ alkyl, (CH₂)ᵣ—C(═O)—NR⁶³R⁶⁴, —C(═O)—(CH₂)ᵣ—NR⁶³R⁶⁴, and C(═O)—X—R⁶⁵;
R⁶³ and R⁶⁴ are independently selected from the group consisting of H, C₁-C₈ alkyl, C₂-C₈ alkenyl, C₂-C₈ alkynyl, —(C₁-C₆ alkyl)-O—(C₁-C₆ alkyl), —(C₁-C₆ alkyl)-NR⁶⁶R⁶⁷, —(C₁-C₆ alkyl)-C(═O)NR⁶⁶R⁶⁷, aryl, aralkyl, heteroaryl, C₃-C₇ cycloalkyl, a three to twelve membered heterocyclic ring containing up to 3 heteroatoms, each of which may be optionally substituted by from 1 to 3 substituents independently selected from halo, C₁-C₆ alkyl, C₂-C₆ alkenyl, C₃-C₇ cycloalkyl, C₁-C₆ alkoxy, hydroxy, amino, cyano and C₁-C₃ perfluoro alkyl;
or R⁶³ and R⁶⁴ may be taken together to form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆alkoxy, C₃-C₇ cycloalkyl, oxo, hydroxy, amino, cyano and C₁-C₃ perfluoro alkyl;
R⁶⁵ is selected from the group consisting of H, aryl, —(C₁-C₆ alkyl)-O—(C₁-C₆ alkyl), (C₁-C₆ alkyl)-NR⁶⁶R⁶⁷, —CO₂R, —O—(CH₂), —CO₂R⁶⁸, and C(═O)NR⁶⁶R⁶⁷
R⁶⁶ and R⁶⁷ independently selected from the group consisting of H, C₁-C₅ alkyl, C₂-C₈ alkenyl, C₂-C₈ alkynyl, —(C₁-C₆ alkyl)-O—(C₁-C₆ alkyl), aryl, aralkyl, heteroaryl, C₃-C₇ cycloalkyl, a three to twelve membered heterocyclic ring containing up to 3 heteroatoms, each of which may be optionally substituted by from 1 to 3 substituents independently selected from halo, C₁-C₆ alkyl, C₂-C₆ alkenyl, C₁-C₆ alkoxy, hydroxy, amino, cyano and C₁-C₃ perfluoro alkyl;
or R⁶⁶ and R⁶⁷ nay be taken together to form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆alkoxy, oxo, hydroxy, amino, cyano and C₁-C₃ perfluoro alkyl;
R⁶⁸ is selected from the group consisting of H, aryl, aralkyl, heteroaryl, C₁-C₆ alkyl, —(C₁-C₆ alkyl)-O—(C₁-C₆ alkyl), —(C₁-C₆ alkyl)-NR⁶⁶R⁶⁷, (C₁-C₆ alkyl)-O—(C₁-C₆alkyl)-O—(C₁-C₆ alkyl), each of which may be optionally substituted by from 1 to 3 substituents independently selected from halo, C₁-C₆ alkoxy, hydroxy, amino, cyano and C₁-C₃ perfluoroalkyl;
r is selected from 0 to 6;
s is selected from 0 to 6;
n is selected from 0 to 4;
m is selected from 0 to 3; and
p is selected from 0 and 1.

In some embodiments, the selective inhibitor of ROCK2 is a compound according to Formula XIV:

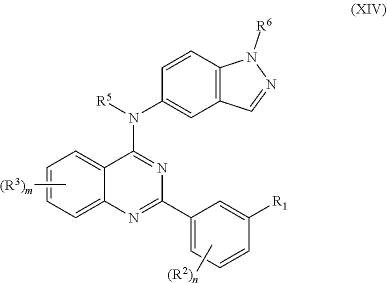

or a pharmaceutically acceptable salt, ester, stereoisomer, polymorph, solvate, N-oxide, or isotopically labeled compound thereof, wherein R¹, R², R³, R⁵, R⁶, m and n are as defined above in Formula XIII.

In some embodiments, the selective inhibitor of ROCK2 is a compound according to Formula XV:

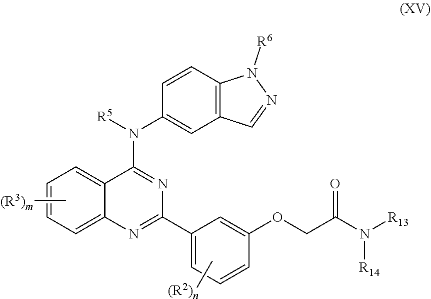

or a pharmaceutically acceptable salt, ester, stereoisomer, polymorph, solvate, N-oxide, or isotopically labeled compound thereof, wherein
R¹³ and R¹⁴ are independently selected from the group consisting of H, C₁-C₈ alkyl, C₂-C₈ alkenyl, C₂-C₈ alkynyl, —(C₁-C₆ alkyl)-O—(C₁-C₆ alkyl), —(C₁-C₆ alkyl)-NR¹⁶R¹⁷, —(C₁-C₆ alkyl)-C(═O)NR¹⁶R¹⁷, aryl, aralkyl, heteroaryl, C₃-C₇ cycloalkyl, a three to twelve membered heterocyclic ring containing up to 3 heteroatoms, each of which may be optionally substituted by from 1 to 3 substituents independently selected from halo, C₁-C₆ alkyl, C₂-C₆ alkenyl, C₃-C₇ cycloalkyl, C₁-C₆ alkoxy, hydroxy, amino, cyano and C₁-C₃ perfluoro alkyl;
or R¹³ and R¹⁴ may be taken together to form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆alkoxy, C₃-C₇ cycloalkyl, oxo, hydroxy, amino, cyano and C₁-C₃ perfluoro alkyl;
R¹⁶ and R¹⁷ are independently selected from the group consisting of H, C₁-C₈ alkyl, C₂-C₈ alkenyl, C₂-C₈ alkynyl, —(C₁-C₆ alkyl)-O—(C₁-C₆ alkyl), aryl, aralkyl, heteroaryl, C₃-C₇ cycloalkyl, a three to twelve membered heterocyclic ring containing up to 3 heteroatoms, each of which may be optionally substituted by from 1 to 3 substituents independently selected from halo, C₁-C₆ alkyl, C₂-C₆ alkenyl, C₁-C₆ alkoxy, hydroxy, amino, cyano and C₁-C₃ perfluoro alkyl;
or R¹⁶ and R¹⁷ may be taken together to form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, oxo, hydroxy, amino, cyano and C₁-C₃ perfluoro alkyl; and
wherein each of R², R³, R⁵, R⁶, m and n are as defined above in Formula XIII.

In some embodiments, the selective inhibitor of ROCK2 is a compound according to Formula XVI:

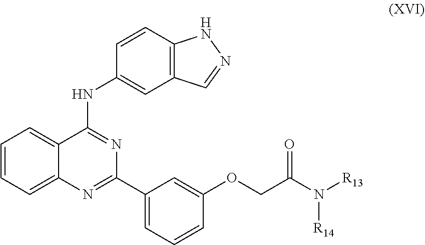

or a pharmaceutically acceptable salt, ester, stereoisomer, polymorph, solvate, N-oxide, or isotopically labeled compound thereof, wherein R¹³ and R¹⁴ are as defined above in Formula XV.

In some embodiments, the selective inhibitor of ROCK2 is (2-(3-(4-((1H-indazol-5-yl)amino)quinazolin-2-yl)phenoxy)-N-isopropylacetamide) having the chemical Formula XVII:

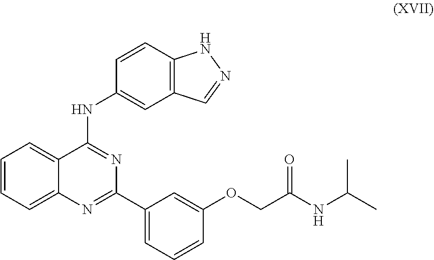 [SMILES: CC(C)NC(=O)COc1cccc(-c2nc(Nc3ccc4[nH]ncc4c3)c3ccccc3n2)c1]

or a pharmaceutically acceptable salt, ester, stereoisomer, polymorph, solvate, N-oxide, or isotopically labeled compound thereof. In some embodiments, the selective inhibitor of ROCK2 is belumosudil.

Compounds of the Formula XIII to XVII, and particularly (2-(3-(4-((1H-indazol-5-yl)amino)quinazolin-2-yl)phenoxy)-N-isopropylacetamide), may be prepared according to the methods disclosed in WO 2014/055996, the contents of which is incorporated herein in its entirety.

In some embodiments, the selective inhibitor of ROCK2 is a ribonucleic acid (RNA). In some embodiments, the RNA is an antisense RNA against ROCK2 transcription. In some embodiments, the RNA is a small interfering RNA (siRNA) or micro RNA (miRNA) that selectively targets an mRNA encoding ROCK2.

As used herein, the symbol [symbol] when used as part of a molecular structure refers to a single bond or a trans or cis double bond.

As used herein, the term “aliphatic” as used herein refers to a straight chained or branched alkyl, alkenyl or alkynyl. It is understood that alkenyl or alkynyl embodiments need at least two carbon atoms in the aliphatic chain. Aliphatic groups typically contain from 1 (or 2) to 12 carbons, such as from 1 (or 2) to 4 carbons.

As used herein, the term “alkylene” refers to a saturated divalent hydrocarbyl, preferably refers to a saturated divalent hydrocarbyl having 1, 2, 3, 4, 5 or 6 carbon atoms, e.g., methylene (—CH₂—), ethylene (—CH₂CH₂—), propylene or butylene.

As used herein, the term “alkyl” is defined as a linear or branched saturated aliphatic hydrocarbon. In some embodiments, alkyl has 1-12, e.g., 1-6, carbon atoms. For example, as used herein, the term “C₁₋₆ alkyl” refers to a linear or branched group having 1-6 carbon atoms (such as methyl, ethyl, n-propyl, isopropyl, n-butyl, isobutyl, sec-butyl, tent-butyl, n-pentyl, isopentyl, neopentyl, or n-hexyl), which is optionally substituted with one or more (e.g., 1 to 3) suitable substituents such as halogen (in which case the group may be referred to as “haloalkyl”) (e.g., CH₂F, CHF₂, CF₃, CCl₃, C₂F₅, C₂Cl₅, CH₂CF₃, CH₂Cl or CH₂CH₂CF₃, etc.). The term “C₁₋₄ alkyl” refers to a linear or branched aliphatic hydrocarbon chain having 1-4 carbon atoms (i.e., methyl, ethyl, n-propyl, isopropyl, n-butyl, isobutyl, sec-butyl or tent-butyl).

As used herein, the term “alkenyl” refers to a linear or branched monovalent hydrocarbyl having a double bond and 2-6 carbon atoms (“C₂₋₆ alkenyl”). The alkenyl is e.g., vinyl, 1-propenyl, 2-propenyl, 2-butenyl, 3-butenyl, 2-pentenyl, 3-pentenyl, 4-pentenyl, 2-hexenyl, 3-hexenyl, 4-hexenyl, 5-hexenyl, 2-methyl-2-propenyl and 4-methyl-3-pentenyl. When the compound of the present invention contains an alkenylene group, the compound may exist as the pure E (entgegen) form, the pure Z (zusammen) form, or any mixture thereof.

As used herein, the term “alkynyl” refers to a monovalent hydrocarbyl containing one or more triple bond, and preferably having 2, 3, 4, 5 or 6 carbon atoms, e.g., ethynyl or propynyl.

The term “cycloalkyl” or “cycloalkenyl” refers to a monocyclic or fused or bridged bicyclic carbocyclic ring system that is not aromatic. For example, cycloalkyl, or cycloalkenyl as used herein can be a C3-C10 monocyclic or fused or bridged C8-C12 bicyclic carbocyclic ring system that is not aromatic (such as bicyclo [1.1.1] pentyl, bicyclo [2.2.1]heptyl, bicyclo [3.2.1] octyl or bicyclo [5 0.2.0] nonyl, or decahydronaphthalene etc.)) which is optionally substituted with one or more (e.g., 1 to 3) suitable substituents. Cycloalkenyl rings have one or more 5 units of unsaturation. Preferred cycloalkyl or cycloalkenyl groups include cyclopropyl, cyclobutyl, cyclopentyl, cyclohexyl, cyclohexenyl, cycloheptyl, cycloheptenyl, norbornyl, adamantyl and decalinyl.

As used herein, the terms “cyclic hydrocarbylene”, “cyclic hydrocarbyl” and “hydrocarbon ring” refer to both saturated (i.e., “cycloalkylene” and “cycloalkyl”) or unsaturated (i.e., having one or more double and/or triple bonds in the ring) monocyclic or polycyclic hydrocarbon ring having e.g., 3-10 (suitably having 3-8, and more suitably having 3-6) ring carbon atoms, including but not limited to cyclopropyl(ene) (ring), cyclobutyl(ene) (ring), cyclopentyl(ene) (ring), cyclohexyl(ene) (ring), cycloheptyl(ene) (ring), cyclooctyl(ene) (ring), cyclononyl(ene) (ring), cyclohexenyl(ene) (ring), and the like.

As used herein, the terms “heterocyclyl”, “heterocyclylene” and “heterocycle” refer to a saturated (i.e., heterocycloalkyl) or partially unsaturated (i.e., having one or more double and/or triple bonds in the ring) cyclic group having e.g. 3-10 (suitably having 3-8, and more suitably having 3-6) ring atoms, wherein at least one ring atom is a heteroatom selected from the group consisting of N, O and S, and the remaining ring atoms are C. For example, “3- to 10-membered heterocyclyl(ene)” of “3- to 10-membered heterocycle” refers to saturated or partially unsaturated heterocyclyl(ene) or heterocycle having 2-9 (e.g., 2, 3, 4, 5, 6, 7, 8 or 9) ring carbon atoms and one or more (e.g., 1, 2, 3, or 4) heteroatoms independently selected from the group consisting of N, O and S. Examples of heterocyclylene, heterocyclyl and heterocycle include, but are not limited to oxiranyl(ene), aziridinyl(ene), azetidinyl(ene), oxetanyl(ene), tetrahydrofuranyl(ene), dioxolinyl(ene), pyrrolidinyl(ene), pyrrolidonyl(ene), imidazolidinyl(ene), pyrazolidinyl(ene), pyrrolinyl(ene), tetrahydropyranyl(ene), piperidinyl(ene), morpholinyl(ene), dithianyl(ene), thiomorpholinyl(ene), piperazinyl(ene) or trithianyl(ene). Said group also encompasses a bicyclic system, including a spiro, fused, or bridged system (e.g., 8-azaspiro[4.5]decane, 3,9-diazaspiro[5.5]undecane, 2-azabicyclo[2.2.2]octane, etc.). Heterocyclylene, heterocyclyl and heterocycle may optionally be substituted with one or more (e.g. 1, 2, 3 or 4) suitable substituents.

The term “aryl” as used herein refers to a monocyclic or bicyclic carbocyclic aromatic ring system having a conjugated π electron system. Aryl as used herein includes a (C6-C12)-aryl-. For example, aryl as used herein can be a C6-C10 monocyclic or C8-C12 bicyclic carbocyclic aromatic ring system. In some embodiments, aryl as used herein can be a (C6-C10)-aryl-. Phenyl (or Ph) is an example of a monocyclic aromatic ring system. Bicyclic aromatic ring systems include 10 systems wherein both rings are aromatic, e.g., naphthyl, and systems wherein only one of the two rings is aromatic, e.g., tetralin. Aryl(ene) or aromatic ring is optionally substituted with one or more (such as 1 to 3) suitable substituents (e.g., halogen, —OH, —CN, —NO₂, and C₁₋₆ alkyl, etc.).

As used herein, the terms “heteroaryl(ene)” and “heteroaromatic ring” refer to a monocyclic, bicyclic or tricyclic aromatic ring system having 5, 6, 8, 9, 10, 11, 12, 13 or 14 ring atoms, particularly 1 or 2 or 3 or 4 or 5 or 6 or 9 or 10 carbon atoms, and containing at least one heteroatom (such as O, N, or S), which can be same to different. Moreover, in each case, it can be benzo-fused. In particular, “heteroaryl(ene)” or “heteroaromatic ring” is selected from the group consisting of thienyl(ene), furyl(ene), pyrrolyl(ene), oxazolyl(ene), thiazolyl(ene), imidazolyl(ene), pyrazolyl(ene), isoxazolyl(ene), isothiazolyl(ene), oxadiazolyl(ene), triazolyl(ene), thiadiazolyl(ene) etc., and benzo derivatives thereof; or pyridinyl(ene), pyridazinyl(ene), pyrimidinyl(ene), pyrazinyl(ene), triazinyl(ene), etc., and benzo derivatives thereof.

As used herein, the term “aralkyl” preferably means aryl or heteroaryl substituted alkyl, wherein aryl, heteroaryl and alkyl are as defined herein. Normally, the aryl group may have 6-14 carbon atoms, the heteroaryl group may have 5-14 ring atoms, and the alkyl group may have 1-6 carbon atoms. Exemplary aralkyl group includes, but is not limited to, benzyl, phenylethyl, phenylpropyl, phenylbutyl.

As used herein, the term “halo” or “halogen” are defined to include F, Cl, Br, or I.

As used herein, the term “nitrogen containing heterocycle” refers to a saturated or unsaturated monocyclic or bicyclic group having 2, 3, 4, 5, 6, 7, 8, 9, 10, 11, 12 or 13 carbon atoms and at least one nitrogen atom in the ring, which may further optionally comprise one or more (e.g., one, two, three or four) ring members selected from the group consisting of N, O, C═O, S, S═O and S(═O)₂. The nitrogen containing heterocycle is attached to the rest of the molecule through the nitrogen atom and any other ring atom in said nitrogen containing heterocycle. The nitrogen containing heterocycle is optionally benzo-fused and is preferably attached to the rest of the molecule through the nitrogen atom in said nitrogen containing heterocycle and any carbon atom in the fused benzene ring.

As used herein, the term “glycoside” refers to any material with a chemical structure comprising a glycosidic bond between a carbohydrate (sugar) molecule and another carbohydrate or a non-carbohydrate (non-sugar) moiety. A glycosidic bond or glycosidic linkage is a type of covalent bond that joins a carbohydrate (sugar) molecule, for example, via its hemiacetal or hemiketal group, to another molecule. The other molecule may or may not be a carbohydrate. The sugar moiety is generally known as the glycone part of a glycoside. The glycone can consist of a single sugar group (monosaccharide) or several sugar groups (oligosaccharide).

As used herein, the term “substituent” refers to a group replacing a second atom or group such as a hydrogen atom on any molecule, compound or moiety. Suitable substituents include, without limitation, halo, hydroxy, mercapto, oxo, nitro, haloalkyl, alkyl, alkaryl, aryl, aralkyl, alkoxy, thioalkoxy, aryloxy, amino, alkoxycarbonyl, amido, carboxy, alkanesulfonyl, alkylcarbonyl, and cyano groups.

As used herein, the term “derivative” refers to a compound that retains the biological activity of the parent compound from which it is derived or is a prodrug for the parent compound. Derivatives may include esters, amides, ethers of the parent compound, obtained by chemically modifying a moiety of the parent compound. For example, a derivative can be a compound in which a hydrogen atom or a certain atomic group is replaced with another atom or atomic group.

The term “substituted” means that one or more (e.g., one, two, three, or four) hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds.

If a substituent is described as being “optionally substituted,” the substituent may be either (1) not substituted, or (2) substituted. If a carbon of a substituent is described as being optionally substituted with one or more of a list of substituents, one or more of the hydrogens on the carbon (to the extent there are any) may separately and/or together be replaced with an independently selected optional substituent. If a nitrogen of a substituent is described as being optionally substituted with one or more of a list of substituents, one or more of the hydrogens on the nitrogen (to the extent there are any) may each be replaced with an independently selected optional substituent.

If substituents are described as being “independently selected” from a group, each substituent is selected independent of the other(s). Each substituent therefore may be identical to or different from the other substituent(s).

As used herein, the term “one or more” means one or more than one (e.g., 2, 3, 4, 5 or 10) as reasonable.

As used herein, unless specified, the point of attachment of a substituent can be from any suitable position of the substituent.

When a bond to a substituent is shown to cross a bond connecting two atoms in a ring, then such substituent may be bonded to any of the ring-forming atoms in that ring that are substitutable.

The compounds for use in the methods provided herein include pharmaceutically acceptable isotopically labeled compounds, which are identical to those of Formulae I to XVII, except that one or more atoms are replaced by an atom having the same atomic number, but an atomic mass or mass number different from the atomic mass or mass number which predominates in nature. Examples of isotopes suitable for inclusion in the compounds include, but are not limited to, isotopes of hydrogen, such as ²H, ³H; carbon, such as ¹¹C, ¹³C, and ¹⁴C; chlorine, such as ³⁶Cl; fluorine, such as ¹⁸F; iodine, such as ¹²³I and ¹²⁵I; nitrogen, such as ¹³N and ¹⁵N; oxygen, such as ¹⁵O, ¹⁷O, and ¹⁸O; phosphorus, such as ³²P; and sulfur, such as ³⁵S. Certain isotopically labeled compounds of the present invention, for example those incorporating a radioactive isotope, are useful in drug and/or substrate tissue distribution studies (e.g., assays). The radioactive isotopes tritium, i.e., ³H, and carbon-14, i.e., ¹⁴C, are particularly useful for this purpose in view of their ease of incorporation and ready means of detection. Substitution with positron-emitting isotopes, such as ¹¹C, ¹⁸F, ¹⁵O and ¹³N, can be useful in positron emission tomography (PET) studies for examining substrate receptor occupancy. Isotopically labeled compounds of the present invention can generally be prepared by processes analogous to those described in the accompanying Schemes and/or in the Examples and Preparations, by using an appropriate isotopically labeled reagent in place of the non-labeled reagent previously employed. Pharmaceutically acceptable solvates in accordance with the invention include those wherein the solvent of crystallization may be isotopically substituted, e.g., D₂O, acetone-d₆, or DMSO-d₆.

The term “stereoisomer” refers to isomers with at least one asymmetric center. A compound having one or more (e.g., one, two, three or four) asymmetric centers can give rise to a racemic mixture, single enantiomer, diastereomer mixture and individual diastereomer. Certain individual molecules may exist as geometric isomers (cis/trans). Similarly, the compounds provided herein may exist as a mixture of two or more structurally different forms in rapid equilibrium (generally referred to as tautomer). Typical examples of a tautomer include a keto-enol tautomer, phenol-keto tautomer, nitroso-oxime tautomer, imine-enamine tautomer and the like. It is to be understood that the use of all such isomers and mixtures thereof in any proportion (such as 60%, 65%, 70%, 75%, 80%, 85%, 90%, 95%, 96%, 97%, 98%, and 99%) are encompassed within the scope of the present invention.

The present invention includes the use of all possible crystalline forms or polymorphs of the compound of the present invention, either as a single polymorph, or as a mixture of more than one polymorph, in any ratio.

It also should be understood that, certain compounds as provided herein can be used for the treatment in a free from, or where appropriate, in a form of a pharmaceutically acceptable derivative. In the present invention, the pharmaceutically acceptable derivative includes, but is not limited to a pharmaceutically acceptable salt, ester, solvate, N-oxide, metabolite, or prodrug, which can directly or indirectly provide the compound of the present invention or a metabolite or residue thereof after being administered to a patient in need thereof. Therefore, the compounds provided herein encompass various derivative forms of the compound as mentioned above.

A pharmaceutically acceptable salt of the compounds disclosed herein includes an acid addition salt or a base addition salt. A suitable acid addition salt is formed from an acid which forms a pharmaceutically acceptable salt. Specific examples include acetate, adipate, aspartate, benzoate, besylate, bicarbonate/carbonate, bisulfate/sulfate, borate, camphorsulfonate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts. A suitable base addition salt is formed from a base which forms a pharmaceutically acceptable salt. Specific examples include aluminum, arginine, benzathine, calcium, choline, diethylamine, diolamine, glycine, lysine, magnesium, meglumine, olamine, potassium, sodium, tromethamine and zinc salts.

For a review on suitable pharmaceutically acceptable salts, see “Handbook of Pharmaceutical Salts: Properties, Selection, and Use” by Stahl and Wermuth (Wiley-VCH, 2002).

As used herein, the term “ester” refers to those derived from the compounds of the various formulae provided herein, which include physiologically-hydrolyzable esters (which may be hydrolyzed under physiological conditions to release the compounds of the present invention in the form of free acids or alcohols).

The compounds for use in the methods of the present invention can exist as a solvate (preferably a hydrate), wherein the compound contains a polar solvent, in particular water, methanol or ethanol for example, as a structural element of the crystal lattice of the compound. The amount of the polar solvent, in particular water, may exist in a stoichiometric or non-stoichiometric ratio.

As can be appreciated by a person skilled in the art, not all nitrogen containing heterocycles can form N-oxides since the nitrogen requires an available electron lone-pair for oxidation to the oxide; a person skilled in the art will recognize those nitrogen containing heterocycles which can form N-oxides. A person skilled in the art will also recognize that tertiary amines can form N-oxides. Synthetic methods for the preparation of N-oxides of heterocycles and tertiary amines are well known to a person skilled in the art, and they include the oxidation of heterocycles and tertiary amines with peroxy acids such as peracetic acid and m-chloroperbenzoic acid (MCPBA), hydrogen peroxide, alkyl hydroperoxides such as tent-butyl hydroperoxide, sodium perborate, and dioxiranes such as dimethyldioxirane. These methods for the preparation of N-oxides have been extensively described and reviewed in literatures, see e.g., T. L. Gilchrist, Comprehensive Organic Synthesis, vol. 7, pp 748-750; A. R. Katritzky and A. J. Boulton, Eds., Academic Press; and G W. H. Cheeseman and E. S. G Werstiuk, Advances in Heterocyclic Chemistry, vol. 22, pp 390-392, A. R. Katritzky and A. J. Boulton, Eds., Academic Press.

The compounds described herein may be administered in the form of a prodrug, in which certain derivatives of the compound that may have little or no pharmacological activity itself, can, when administered into or onto the body, be converted into a compound having the desired activity, for example, by hydrolytic cleavage. In general, such prodrug will be a functional derivative of the compound which is readily converted in vivo into the compound with desired therapeutic activity. Further information on the use of the prodrug may be found in “Pro-drugs as Novel Delivery Systems, Vol. 14, ACS Symposium Series (T. Higuchi and V. Stella). The prodrug can, for example, be produced by replacing appropriate functionalities present in the compound of the present invention with moieties known to those skilled in the art as “pro-moieties” as described, for example, in “Design of Prodrugs” by H. Bundgaard (Elsevier, 1985).

In some embodiments, compounds of the Formulas I to XVII are selective inhibitors of Rho-associated coiled-coil kinase 2 (ROCK2) in human cells. Compounds of the Formulas I to XVII, for example as a pharmaceutical composition comprising the compound, are used to treat (i.e., cure or reduce the severity of, etc.) muscular dystrophy, including Duchenne muscular dystrophy, Becker muscular dystrophy, facioscapulohumeral muscular dystrophy, limb-girdle muscular dystrophy, myotonic muscular dystrophy, oculopharyngeal muscular dystrophy, tibial muscular dystrophy, congenital muscular dystrophy, distal muscular dystrophy, and Emery-Dreifuss muscular dystrophy, particularly Duchenne muscular dystrophy.

### Pharmaceutical Compositions

In one aspect, the present disclosure provides pharmaceutically acceptable compositions for use in the treatment of muscular dystrophy which comprise a therapeutically effective amount of one or more of the selective inhibitors of ROCK2 disclosed herein (e.g., compounds of Formulae I to XVII) formulated together with one or more pharmaceutically acceptable carriers.

The selective inhibitors of ROCK2 disclosed herein may be incorporated into pharmaceutical compositions suitable for administration to a subject. Typically, the pharmaceutical composition comprises a selective inhibitor of ROCK2 disclosed herein and a pharmaceutically acceptable carrier. As used herein, “pharmaceutically acceptable carrier” and “pharmaceutically acceptable excipient” are used interchangeably refer to any and all solvents, dispersion media, coatings, antibacterial and antifungal agents, isotonic and absorption delaying agents, and the like that are physiologically compatible. Pharmaceutically acceptable carriers are well known in the art. See, e.g., Remington's Pharmaceutical Sciences and U.S. Pharmacopeia: National Formulary, Mack Publishing Company, Easton, PA (1984), incorporated herein by reference. Some examples of pharmaceutically acceptable carriers are water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Additional examples of pharmaceutically acceptable substances are wetting agents or minor amounts of auxiliary substances such as wetting or emulsifying agents, preservatives, or buffers, which enhance the shelf life or effectiveness of the selective inhibitor of ROCK2. Pharmaceutical compositions may be prepared by mixing an selective inhibitor of ROCK2 disclosed herein with acceptable carriers, excipients, or stabilizers in the form of, e.g., lyophilized powders, slurries, aqueous solutions or suspensions (see, e.g., Hardman, et al. (2001) Goodman and Gilman's The Pharmacological Basis of Therapeutics, McGraw-Hill, New York, NY; Gennaro (2000) Remington: The Science and Practice of Pharmacy, Lippincott, Williams, and Wilkins, New York, NY; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, NY; each incorporated herein by reference).

The pharmaceutical compositions may be in a variety of forms, for example, liquid, semi solid and solid dosage forms, such as liquid solutions (e.g., injectable and infusible solutions), dispersions or suspensions, tablets, pills, powders, liposomes and suppositories. The preferred form depends on the intended mode of administration and therapeutic application. In some embodiments, the pharmaceutical compositions are in the form of injectable or infusible solutions. Optionally, mode of administration is parenteral (e.g., intravenous, subcutaneous, intraperitoneal, intramuscular). The pharmaceutical composition may be administered by intravenous infusion or injection. In some embodiments, the selective inhibitor of ROCK2 is administered by intramuscular or subcutaneous injection. Formulations for injection may be presented in unit dosage form, e.g., in ampoules, pre-filled syringes, or in multi-dose containers, with or without an added preservative. The pharmaceutical compositions may take such forms as suspensions, solutions, or emulsions in oily or aqueous vehicles, and may contain formulatory agents such as suspending, stabilizing and/or dispersing agents. Alternatively, the active ingredient may be prepared in powder form for reconstitution with a suitable vehicle, e.g., sterile pyrogen-free water, before use.

Therapeutic compositions typically must be sterile and stable under the conditions of manufacture and storage. The composition may be formulated as a solution, microemulsion, dispersion, liposome, or other ordered structure suitable to high drug concentration. Sterile injectable solutions may be prepared by incorporating the selective inhibitor of ROCK2 in the required amount in an appropriate solvent with one or a combination of ingredients enumerated above, as required, followed by filtered sterilization. Dispersions may be prepared by incorporating the selective inhibitor of ROCK2 into a sterile vehicle that contains a basic dispersion medium and the required other ingredients from those enumerated above. In the case of sterile powders for the preparation of sterile injectable solutions, the preferred methods of preparation include vacuum drying and freeze drying that yield a powder of the selective inhibitor of ROCK2 and any additional desired ingredient from a previously sterile filtered solution thereof. The proper fluidity of a solution can be maintained, for example, by the use of a coating such as lecithin, by the maintenance of the required particle size in the case of dispersion and/or by the use of surfactants. Prolonged absorption of injectable compositions can be brought about by including in the composition an agent that delays absorption, for example, monostearate salts and gelatin.

The pharmaceutical compositions may be administered by a variety of methods known in the art. In some embodiments, the preferred route/mode of administration is subcutaneous, intramuscular, or intravenous infusion. As will be appreciated by the skilled artisan, the route and/or mode of administration will vary depending upon the desired results.

In some embodiments, the pharmaceutical compositions may be prepared with a carrier that will protect the selective inhibitor of ROCK2 against rapid release, such as a controlled release formulation, including implants, transdermal patches, and microencapsulated delivery systems. Biodegradable, biocompatible polymers, such as ethylene vinyl acetate, polyanhydrides, polyglycolic acid, collagen, polyorthoesters, and polylactic acid, may be used. Methods for the preparation of such formulations are generally known to those skilled in the art. See, e.g., Sustained and Controlled Release Drug Delivery Systems J. R. Robinson, ed., Marcel Dekker, Inc., New York, 1978, which is incorporated herein by reference.

Additional active compounds also can be incorporated into the compositions. In certain embodiments, a selective inhibitor of ROCK2 disclosed herein is co-formulated with and/or co-administered with one or more additional therapeutic agents. These agents include, without limitation, corticosteroids, angiotensin-converting enzyme (ACE) inhibitors, beta blockers, exon skipping oligonucleotides, and gene therapies. Such combination therapies may require lower dosages of the selective inhibitor of ROCK2 as well as the co-administered agents, thus avoiding possible toxicities or complications associated with the various monotherapies.

Dosage regimens may be adjusted to provide the optimum desired response (e.g., a therapeutic or prophylactic response). For example, a single bolus can be administered, several divided doses can be administered over time or the dose can be proportionally reduced or increased as indicated by the exigencies of the therapeutic situation. It may be advantageous to formulate parenteral compositions in dosage unit form for ease of administration and uniformity of dosage. The term “dosage unit form” as used herein refers to physically discrete units suited as unitary dosages for the subjects to be treated; each unit containing a predetermined quantity of active compound calculated to produce the desired therapeutic effect in association with the required pharmaceutical carrier. The specification for the dosage unit forms may be dictated by and directly dependent on (a) the unique characteristics of the selective inhibitor of ROCK2 and the particular therapeutic or prophylactic effect to be achieved, and (b) the limitations inherent in the art of compounding such a selective inhibitor of ROCK2 for the treatment of sensitivity in individuals.

An exemplary, non-limiting range for a therapeutically or prophylactically effective amount of a selective inhibitor of ROCK2 disclosed herein is about 0.0001 to about 100 mg/kg body weight. The therapeutically or prophylactically effective amount of a selective inhibitor of ROCK2 disclosed herein may be 0.0001 to 100 mg/kg body weight. In some embodiments, a therapeutically or prophylactically effective amount of a selective inhibitor of ROCK2 disclosed herein about 0.01 to about 10 mg/kg body weight. The therapeutically or prophylactically effective amount of a selective inhibitor of ROCK2 disclosed herein may be 0.01 to 10 mg/kg body weight. Optionally, the therapeutically or prophylactically effective amount of a selective inhibitor of ROCK2 disclosed herein is about 0.01 to about 5 mg/kg body weight. In some embodiments, the therapeutically or prophylactically effective amount of a selective inhibitor of ROCK2 disclosed herein is 0.01 to 5 mg/kg body weight. In some embodiments, the therapeutically or prophylactically effective amount of a selective inhibitor of ROCK2 disclosed herein is about 0.05 to about 0.5 mg/kg body weight. In some embodiments, the therapeutically or prophylactically effective amount of a selective inhibitor of ROCK2 disclosed herein is 0.05 to 0.5 mg/kg body weight. Dosage values may vary with the type and severity of the condition to be alleviated. For any particular subject, specific dosage regimens may be adjusted over time according to the individual need and the professional judgment of the person administering or supervising the administration of the compositions, and that dosage ranges set forth herein are exemplary only and are not intended to limit the scope or practice of the paragraphed composition.

Combinations with Other Active Agents

The selective inhibitors of ROCK2 disclosed herein may be used in combination with at least one additional muscular dystrophy treatment. The at least one additional muscular dystrophy treatment may be, for example, a corticosteroid, an angiotensin-converting enzyme (ACE) inhibitor, a beta blocker, an exon skipping oligonucleotide, and a gene therapy, or a mixture thereof.

It is also possible to combine any selective inhibitor of ROCK2 of the disclosure with one or more additional muscular dystrophy treatments in a unitary dosage form for simultaneous or sequential administration to a patient. The combination therapy may be administered as a simultaneous or sequential regimen. When administered sequentially, the combination may be administered in two or more administrations.

Co-administration of a selective inhibitor of ROCK2 of the disclosure with one or more additional muscular dystrophy treatments generally refers to simultaneous or sequential administration of a selective inhibitor of ROCK2 of the disclosure and one or more additional muscular dystrophy treatments, such that therapeutically effective amounts of the selective inhibitor of ROCK2 of the disclosure and one or more additional muscular dystrophy treatments are both present in the body of the patient.

In some embodiments, at the time the selective inhibitor of ROCK2 is administered, the subject has previously received one or more additional muscular dystrophy treatments. In some embodiments, at the time the selective inhibitor of ROCK2 is administered, the subject is concurrently receiving one or more additional muscular dystrophy treatments. In some embodiments, at the time the selective inhibitor of ROCK2 is administered, the subject will subsequently receive one or more additional muscular dystrophy treatments.

In some embodiments, the one or more additional muscular dystrophy treatments includes a corticosteroid. In some embodiments, the corticosteroid is prednisone or deflazacort. In some embodiments, the one or more additional muscular dystrophy treatments includes an ACE inhibitor. In some embodiments, the one or more additional muscular dystrophy treatments includes a beta blocker.

In some embodiments, the one or more additional muscular dystrophy treatments includes an exon skipping oligonucleotide. Exon skipping technologies are known in the art. See, e.g., WO2016198676A1, incorporated by reference herein in its entirety. In some embodiments, the exon skipping oligonucleotide is eteplirsen or golodirsen. In some embodiments, the exon skipping oligonucleotide is eteplirsen. In some embodiments, the exon skipping oligonucleotide is golodirsen.

In some embodiments, the one or more additional muscular dystrophy treatments includes a gene therapy. In some embodiments, the gene therapy is PF-06939926. PF-06939926 is an investigational recombinant adeno-associated virus serotype 9 (rAAV9) capsid carrying a shortened version of the human dystrophin gene (mini-dystrophin) under the control of a human muscle-specific promotor. The rAAV9 capsid potentially targets muscle tissue. See, e.g., U.S. 2017/0368198, incorporated by reference herein in its entirety. In some embodiments, the gene therapy is SRP-9001. SRP-9001 (rAAVrh74.MHCK7.micro-dystrophin) is an investigational gene transfer therapy intended to deliver the micro-dystrophin-encoding gene to muscle tissue for the targeted production of the micro-dystrophin protein. In some embodiments, the gene therapy is a Crispr/cas-related compositions targeting the dystrophin gene (see, e.g., WO2017193029A2, incorporated by reference herein in its entirety). In some embodiments, the gene therapy is a hybrid dual AAV (hdAAV) vector system or a hybrid tri-AAV (htAAV) vector system (see, e.g., U.S. Pat. No. 8,236,557, incorporated by reference herein in its entirety).

### Methods of Treating Muscular Dystrophy

In one aspect, the disclosure provides methods for treating muscular dystrophy in a subject in need thereof. In some embodiments, the method comprises administering to the subject a therapeutically effective amount of comprising a selective inhibitor of Rho-associated coiled-coil kinase 2 (ROCK2), or a composition comprising a selective inhibitor of ROCK2. Without being bound by theory, selective inhibitors of ROCK2, such as, for example, compounds of the Formulae I to XVII, may interfere with the replacement of damaged muscle tissues with non-contractile fibrous tissue.

In another aspect, the present disclosure provides any of the selective inhibitors of ROCK2 disclosed herein or any combinations thereof, or a composition comprising one or more of the selective inhibitors of ROCK2 for use in the treatment of muscular dystrophy in a subject in need thereof.

In another aspect, the present disclosure provides the use of any of the selective inhibitors of ROCK2 disclosed herein or any combinations thereof, or a composition comprising one or more of the selective inhibitors of ROCK2 in the manufacture of a medicament for the treatment of muscular dystrophy in a subject in need thereof.

In some embodiments, the muscular dystrophy is selected from the group consisting of Duchenne muscular dystrophy, Becker muscular dystrophy, facioscapulohumeral muscular dystrophy, limb-girdle muscular dystrophy, myotonic muscular dystrophy, oculopharyngeal muscular dystrophy, tibial muscular dystrophy, congenital muscular dystrophy, distal muscular dystrophy, and Emery-Dreifuss muscular dystrophy. In some embodiments, the muscular dystrophy is Duchenne muscular dystrophy. In some embodiments, the muscular dystrophy is Becker muscular dystrophy. In some embodiments, the muscular dystrophy is facioscapulohumeral muscular dystrophy. In some embodiments, the muscular dystrophy is limb-girdle muscular dystrophy. In some embodiments, the muscular dystrophy is myotonic muscular dystrophy. In some embodiments, the muscular dystrophy is oculopharyngeal muscular dystrophy. In some embodiments, the muscular dystrophy is tibial muscular dystrophy. In some embodiments, the muscular dystrophy is congenital muscular dystrophy. In some embodiments, the muscular dystrophy is distal muscular dystrophy. In some embodiments, the muscular dystrophy is Emery-Dreifuss muscular dystrophy.

In some embodiments, the selective inhibitor of ROCK2 is a compound according to Formula I, as defined herein, or a pharmaceutically acceptable salt, ester, stereoisomer, polymorph, solvate, N-oxide, or isotopically labeled compound thereof. In some embodiments, the selective inhibitor of ROCK2 is a compound according to any one of Formula II to IX, as defined herein, or a pharmaceutically acceptable salt, ester, stereoisomer, polymorph, solvate, N-oxide, or isotopically labeled compound thereof. In some embodiments, the selective inhibitor of ROCK2 is a compound according to Formula X or XI, as defined herein, or a pharmaceutically acceptable salt, ester, stereoisomer, polymorph, solvate, N-oxide, or isotopically labeled compound thereof. In some embodiments, the selective inhibitor of ROCK2 is a compound according to Formula XII, as defined herein, or a pharmaceutically acceptable salt, ester, stereoisomer, polymorph, solvate, N-oxide, or isotopically labeled compound thereof, and particularly (6-(4-((4-(1H-pyrazol-4-yl)phenyl)amino)pyrimidin-2-yl)-1-methyl-1H-indol-2-yl)(3,3-difluoroazetidin-1-yl)methanone.

In some embodiments, the selective inhibitor of ROCK2 is a compound according to Formula XIII, as defined herein, or a pharmaceutically acceptable salt, ester, stereoisomer, polymorph, solvate, N-oxide, or isotopically labeled compound thereof. In some embodiments, the selective inhibitor of ROCK2 is a compound according to Formula XIV, as defined herein, or a pharmaceutically acceptable salt, ester, stereoisomer, polymorph, solvate, N-oxide, or isotopically labeled compound thereof. In some embodiments, the selective inhibitor of ROCK2 is a compound according to Formula XV, as defined herein, or a pharmaceutically acceptable salt, ester, stereoisomer, polymorph, solvate, N-oxide, or isotopically labeled compound thereof. In some embodiments, the selective inhibitor of ROCK2 is a compound according to Formula XVI, as defined herein, or a pharmaceutically acceptable salt, ester, stereoisomer, polymorph, solvate, N-oxide, or isotopically labeled compound thereof. In some embodiments, the selective inhibitor of ROCK2 is a compound according to Formula XVII, as defined herein, or a pharmaceutically acceptable salt, ester, stereoisomer, polymorph, solvate, N-oxide, or isotopically labeled compound thereof, and particularly (2-(3-(4-((1H-indazol-5-yl)amino)quinazolin-2-yl)phenoxy)-N-isopropylacetamide).

In some embodiments, the selective inhibitor of ROCK2 is a ribonucleic acid (RNA). The RNA may be an antisense RNA against ROCK2 transcription. In some embodiments, the RNA is a small interfering RNA (siRNA) or micro RNA (miRNA).

In some embodiments, the subject is a human. In some embodiments, the human subject is an adult patient. In some embodiments, the human subject is a pediatric patient. In embodiments, the human subject suffers from muscular dystrophy. In embodiments, the human subject is at risk of developing muscular dystrophy.

In some embodiments, the subject has previous been administered an additional muscular dystrophy treatment. In some embodiments, the subject is concurrently being administered an additional muscular dystrophy treatment. In some embodiments, the subject will subsequently be administered an additional muscular dystrophy treatment.

In some embodiments, the additional muscular dystrophy treatment is selected from the group consisting of a corticosteroid, an angiotensin-converting enzyme (ACE) inhibitor, a beta blocker, an exon skipping oligonucleotide, and a gene therapy. In some embodiments, the corticosteroid is prednisone or deflazacort. In some embodiments, the exon skipping oligonucleotide is eteplirsen or golodirsen. In some embodiments, the gene therapy is SRP-9001 or PF-06939926.

One or more selective inhibitors of ROCK2 of the disclosure are administered by any route appropriate to the condition to be treated. Non-limiting examples of suitable routes include oral and parenteral (including subcutaneous, intramuscular, intravenous, intradermal, intrathecal and epidural), and the like. It can be appreciated that the preferred route may vary with for example the condition of the recipient. An advantage of the selective inhibitors of ROCK2 of this disclosure is that they are orally bioavailable and can be dosed orally.

An effective dose of the selective inhibitors of ROCK2 of the disclosure depends at least on the nature of the muscular dystrophy being treated, toxicity, whether the selective inhibitor of ROCK2 is being used prophylactically or therapeutically, the method of delivery, and the pharmaceutical formulation, and can be determined by the clinician using conventional dose escalation studies. In some embodiments, doses of the selective inhibitors of ROCK2 of the disclosure range from about 0.1 to about 50 mg/kg body weight. The dose for adult human may range from 1 mg to 1000 mg, for example between about 5 mg and about 800 mg or between about 50 mg and 500 mg and may take the form of single or multiple doses. In embodiments, the dose of a compound of Formulas I-XVII to treat or prevent muscular dystrophy is about 10 mg, about 20 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, or about 800 mg, which may be administered as a single daily dose, or split between two, three or more administrations. In embodiments, the dose is about 400 mg per day BID.

### Kits

In one aspect, the present disclosure provides a kit or composition that includes a selective inhibitor of ROCK2 of the disclosure (e.g. a compound of Formulae I to XVII, or a pharmaceutically acceptable salt, pharmaceutically acceptable ester, stereoisomer, mixture of stereoisomers or tautomer thereof). In some embodiments individual kits are provided comprising a compound of any one of Formulae I to XVII, or a pharmaceutically acceptable salt, pharmaceutically acceptable ester, stereoisomer, mixture of stereoisomers or tautomer thereof. In some embodiments, the kit comprises a compound of any one of Formulae I to XVII, or a pharmaceutically acceptable salt thereof.

Each of the individual kits described herein may comprise a label and/or instruction for use of the compound in the treatment of a muscular dystrophy in a subject (e.g., human) in need thereof. In some embodiments, the muscular dystrophy is selected from the group consisting of Duchenne muscular dystrophy, Becker muscular dystrophy, facioscapulohumeral muscular dystrophy, limb-girdle muscular dystrophy, myotonic muscular dystrophy, oculopharyngeal muscular dystrophy, tibial muscular dystrophy, congenital muscular dystrophy, distal muscular dystrophy, and Emery-Dreifuss muscular dystrophy. In some embodiments, the muscular dystrophy is Duchenne muscular dystrophy. In some embodiments, the muscular dystrophy is Becker muscular dystrophy. In some embodiments, the muscular dystrophy is facioscapulohumeral muscular dystrophy. In some embodiments, the muscular dystrophy is limb-girdle muscular dystrophy. In some embodiments, the muscular dystrophy is myotonic muscular dystrophy. In some embodiments, the muscular dystrophy is oculopharyngeal muscular dystrophy. In some embodiments, the muscular dystrophy is tibial muscular dystrophy. In some embodiments, the muscular dystrophy is congenital muscular dystrophy. In some embodiments, the muscular dystrophy is distal muscular dystrophy. In some embodiments, the muscular dystrophy is Emery-Dreifuss muscular dystrophy.

In some embodiments, each kit may also contain instructions for use of additional muscular dystrophy treatment in combination with selective inhibitor of ROCK2 (e.g. a compound of any one of Formulae I to XVII or a pharmaceutically acceptable salt, pharmaceutically acceptable ester, stereoisomer, mixture of stereoisomers or tautomer thereof) in the treatment of muscular dystrophy in a subject (e.g., human) in need thereof. In some embodiments, the kit comprises individual dose units of a compound described herein. Examples of individual dosage units may include pills, tablets, capsules, prefilled syringes or syringe cartridges, IV bags, etc., each comprising a therapeutically effective amount of the selective inhibitor of ROCK2 in question, or a pharmaceutically acceptable salt, racemate, enantiomer, diastereomer, tautomer, polymorph, pseudopolymorph, amorphous form, hydrate or solvate thereof. In some embodiments, the kit may contain a single dosage unit and in others multiple dosage units are present, such as the number of dosage units required for a specified regimen or period.

Also provided are articles of manufacture that include a selective inhibitor of ROCK2 (e.g. a compound of any one of Formulas I to XVII, or a pharmaceutically acceptable salt, pharmaceutically acceptable ester, stereoisomer, mixture of stereoisomers or tautomer thereof); and a container. In some embodiments, the article of manufacture comprises a compound of any one of Formulas I to XVII, or a pharmaceutically acceptable salt thereof, and a container. In some embodiments, the container of the article of manufacture is a vial, jar, ampoule, preloaded syringe, blister package, tin, can, bottle, box, or an intravenous bag.

### Numbered Embodiments

Particular embodiments of the disclosure are set forth in the following numbered embodiments:
1. A method for treating muscular dystrophy in a subject in need thereof, the method comprising administering to the subject a therapeutically effective amount of a selective inhibitor of Rho-associated coiled-coil kinase 2 (ROCK2).
2. The method of embodiment 1, wherein the muscular dystrophy is selected from the group consisting of Duchenne muscular dystrophy, Becker muscular dystrophy, facioscapulohumeral muscular dystrophy, limb-girdle muscular dystrophy, myotonic muscular dystrophy, oculopharyngeal muscular dystrophy, tibial muscular dystrophy, congenital muscular dystrophy, distal muscular dystrophy, and Emery-Dreifuss muscular dystrophy.
3. The method of embodiment 2, wherein the muscular dystrophy is Duchenne muscular dystrophy.
4. The method of any one of embodiments 1-3, wherein the selective inhibitor of ROCK2 is compound according to Formula I:

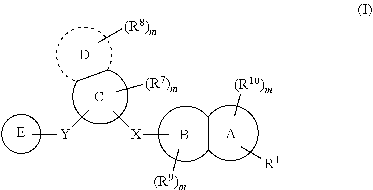

or a pharmaceutically acceptable salt, ester, stereoisomer, polymorph, solvate, N-oxide, or isotopically labeled compound thereof, wherein:
   X and Y are each independently selected from the group consisting of a direct bond, C(═O), O, S(═O)ᵢ and NR;
   R is selected from the group consisting of H, C₁₋₆ alkyl, C₂₋₆ alkenyl, C₂₋₆ alkynyl, saturated or partially unsaturated C₃₋₁₀ cyclic hydrocarbyl, saturated or partially unsaturated 3- to 10-membered heterocyclyl, C₆₋₁₀ aryl, 5- to 14-membered heteroaryl and C₆₋₁₂ aralkyl, and at most 2 ring members in the cyclic hydrocarbyl and heterocyclyl are C(═O);
   ring A and ring B are each independently selected from the group consisting of saturated or partially unsaturated C₃₋₁₀ hydrocarbon ring, saturated or partially unsaturated 3- to 10-membered heterocycle, C₆₋₁₀ aromatic ring and 5- to 14-membered heteroaromatic ring, and at most 2 ring members in the hydrocarbon ring and heterocycle are C(═O); provided that when ring B is a heterocycle containing a nitrogen atom, ring B is not attached to X via the nitrogen atom;
   ring C is selected from the group consisting of saturated or partially unsaturated C₃₋₁₀ hydrocarbon ring, saturated or partially unsaturated 3- to 10-membered heterocycle, C₆₋₁₀ aromatic ring and 5- to 14-membered heteroaromatic ring, and at most 2 ring members in the hydrocarbon ring and heterocycle are C(═O);
   ring D is absent, or is selected from the group consisting of saturated or partially unsaturated C₃₋₁₀ hydrocarbon ring, saturated or partially unsaturated 3- to 10-membered heterocycle, C₆₋₁₀ aromatic ring and 5- to 14-membered heteroaromatic ring, and at most 2 ring members in the hydrocarbon ring and heterocycle are C(═O);
   ring E is selected from the group consisting of:

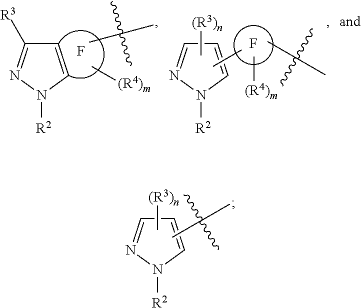

ring F is selected from the group consisting of saturated or partially unsaturated C₃₋₁₀ hydrocarbon ring, saturated or partially unsaturated 3- to 10-membered heterocycle, C₆₋₁₀ aromatic ring and 5- to 14-membered heteroaromatic ring, and at most 2 ring members in the hydrocarbon ring and heterocycle are C(═O);
R¹ is selected from the group consisting of H, —NH₂, C₁₋₆ alkyl, C₆₋₁₀ aryl, 5- to 14-membered heteroaryl, N-methylpyrrolidinyl, N-methylpiperidinyl,

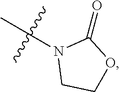 [SMILES: *N1CCOC1=O]

acetyl,

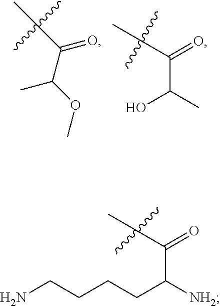

—C(═O)—(C₁₋₆ alkylene)n-CF₃, —C(═O)—(C₁₋₆ alkylene) CN, —C(═O)-(saturated or partially unsaturated C₃₋₁₀ cyclic hydrocarbyl), —NHC(═O)-(saturated or partially unsaturated C₃₋₁₀ cyclic hydrocarbyl), —C(═O)-(saturated or partially unsaturated 3- to 10-membered heterocyclyl), —C(═O)—C₁₋₆ alkylene-(saturated or partially unsaturated 3- to 10-membered heterocyclyl), —C(═O)-(5- to 14-membered heteroaryl), —C(═O)—C₁₋₆ alkylene-NH(C₁₋₆ alkyl), —C(═O)—C₁₋₆ alkylene-N(C₁₋₆ alkyl)₂, N-methylpiperazine substituted acetyl, —S(═O)₂R^{1a}, —P(═O)R^{1a}R^{1b},

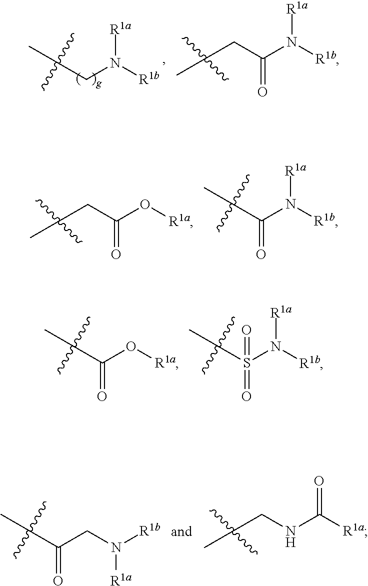

provided that when one of R¹ and R¹⁰ is C¹⁻⁶ alkyl, and the other is H or C³⁻¹⁰ cyclic hydrocarbyl, at least one of X and Y is a direct bond, and ring C is not a 5-membered heteroaromatic ring; when one of R¹ and R¹⁰ is H, and the other is

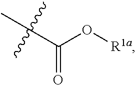

ring C is not a 5-membered heteroaromatic ring; when both R¹ and R¹⁰ are H, ring A contains at least one nitrogen atom, and is not a 5- or 6-membered ring; when one of R¹ and R¹⁰ is H, and the other is

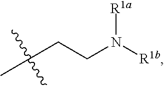

ring C is not a 5-membered heteroaromatic ring; and when one of R¹ and R¹⁰ is H, and the other is H or acetyl, ring D is absent;
R^{1a} and R^{1b} are each independently selected from the group consisting of H, halogen, amino, cyano, nitro, C₁₋₆ alkyl, C₂₋₆ alkenyl, C₂₋₆ alkynyl, C₃₋₁₀ cyclic hydrocarbyl, 3- to 10-membered heterocyclyl, C₆₋₁₀ aryl, 5- to 14-membered heteroaryl, C₆₋₁₂ aralkyl,
—C(═O)R⁵, —OC(═O)R⁵, —C(═O)OR⁵, —OR⁵, —SR⁵, —S(═O)R⁵, —S(═O)₂R⁵,
—S(═O)₂NR⁵R⁶, —NR⁵R⁶, —C(═O)NR⁵R⁶, —NR⁵—C(═O)R⁶, —NR⁵—C(═O)OR⁶,
NR⁵—S(═O)₂—R⁶, —NR⁵—C(═O)—NR⁵R⁶, —C₁₋₆ alkylene-NR⁵R⁶, —C₁₋₆ alkylene-OR⁵ and
—O—C₁₋₆ alkylene-NR⁵R⁶, provided that when one of R^{1a} and R^{1b} is n-propyl, the other is not H; or R^{1a} and R^{1b} together with the atom to which they are attached form a 3- to 12-membered heterocycle or heteroaromatic ring;
R², R³, R⁴, R⁷, R⁸, R⁹ and R¹⁰, at each occurrence, are each independently selected from the group consisting of H, halogen, amino, cyano, nitro, C₁₋₆ alkyl, C₂₋₆ alkenyl, C₂₋₆ alkynyl, C₃₋₁₀ cyclic hydrocarbyl, 3- to 10-membered heterocyclyl, C₆₋₁₀ aryl, 5- to 14-membered heteroaryl, C₆₋₁₂ aralkyl, —C(═O)R⁵, OC(═O)R⁵, —C(═O)OR⁵, OR⁵, SR⁵, —S(═O)R⁵, —S(═O)₂R⁵, —S(═O)₂NR⁵R⁶, —NR⁵R⁶, —C(═O)NR⁵R⁶, —NR⁵—C(═O)R⁶,
—NR⁵—C(═O)OR⁶, —NR⁵—S(═O)₂—R⁶, —NR⁵—C(═O)—NR⁵R⁶, —C₁₋₆ alkylene-NR⁵R⁶,
—C₁₋₆ alkylene-O(P═O)(OH)₂ and —O—C₁₋₆ alkylene-NR⁵R⁶;
the above alkyl, alkylene, alkenyl, alkynyl, cyclic hydrocarbyl, hydrocarbon ring, heterocyclyl, heterocycle, aryl, aromatic ring, heteroaryl, heteroaromatic ring and aralkyl, at each occurrence, are each optionally substituted with one or more substituents independently selected from the group consisting of halogen, hydroxyl, oxo, amino, cyano, nitro, C₁₋₆ alkyl, C₂₋₆ alkenyl, C₂₋₆ alkynyl, C₃₋₆ cyclic hydrocarbyl, 3- to 10-membered heterocyclyl, C₆₋₁₀ aryl, 5- to 14-membered heteroaryl,
C₆₋₁₂ aralkyl, ═N—OR⁵, —C(═NH)NH₂, —C(═O)R⁵, —OC(═O)R⁵, —C(═O)OR⁵, —OR⁵,
—SR⁵, —S(═O)R⁵, —S(═O)₂R⁵, —S(═O)₂NR⁵R⁶, —NR⁵R⁶, —C(═O)NR⁵R⁶, —NR⁵—C(═O)R⁶, —NR⁵—C(═O)OR⁶, —NR⁵—S(═O)₂—R⁶, —NR⁵—C(═O)—NR⁵R⁶, —C₁₋₆ alkylene-NR⁵R⁶ and
—O—C₁₋₆ alkylene-NR⁵R⁶, and the alkyl, cyclic hydrocarbyl, heterocyclyl, aryl, heteroaryl and aralkyl are further optionally substituted with one or more substituents independently selected from the group consisting of halogen, hydroxyl, oxo, amino, cyano, nitro, C₁₋₆ alkyl, C₃₋₆ cyclic hydrocarbyl, 3- to 10-membered heterocyclyl, C₆₋₁₀ aryl, 5- to 14-membered heteroaryl and C₆₋₁₂ aralkyl;
R⁵ and R⁶, at each occurrence, are each independently selected from the group consisting of H, alkyl, C₃₋₁₀ cyclic hydrocarbyl, 3- to 10-membered heterocyclyl, C₆₋₁₀ aryl, 5- to 14-membered heteroaryl and C₆₋₁₂ aralkyl;
m, at each occurrence, is each independently an integer of 0, 1, 2 or 3;
n is an integer of 0, 1 or 2;
i is an integer of 0, 1 or 2; and
g is an integer of 0, 1, 2, 3 or 4.
5. The method of any one of embodiments 1-4, wherein the selective inhibitor of ROCK2 is a compound according to any one of Formula II to IX:

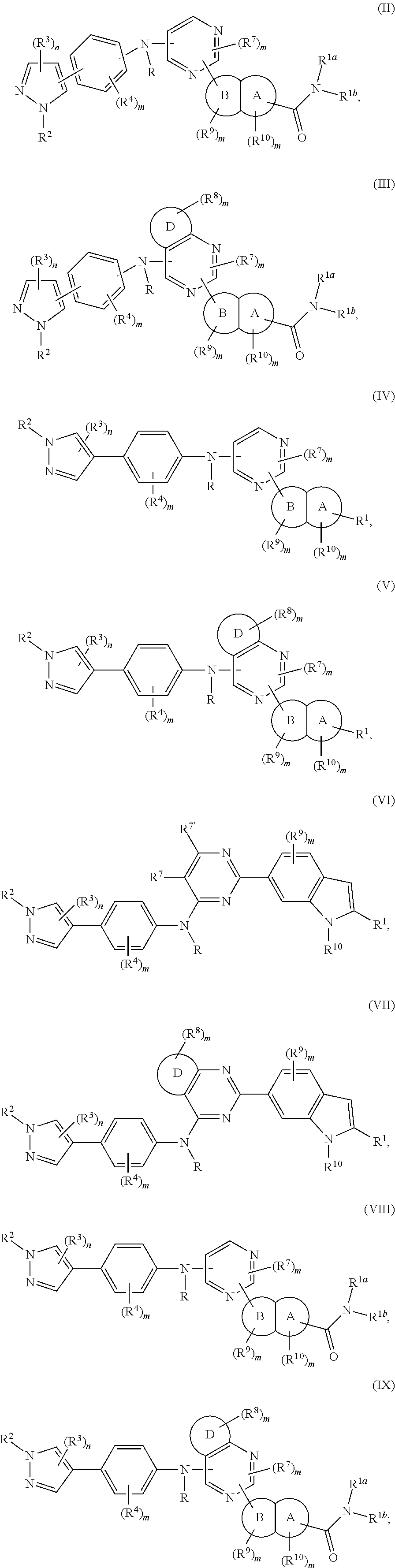

or a pharmaceutically acceptable salt, ester, stereoisomer, polymorph, solvate, N-oxide, or isotopically labeled compound thereof, wherein each of ring A, ring B, ring D, R, R¹, R^{1a}, R^{1b}, R², R³, R⁴, R⁷, R^{7′}, R⁸, R⁹, R¹⁰, n and m are defined for Formula I.
6. The method of any one of embodiments 1-5, wherein the selective inhibitor ROCK2 is a compound according to Formula X or formula XI:

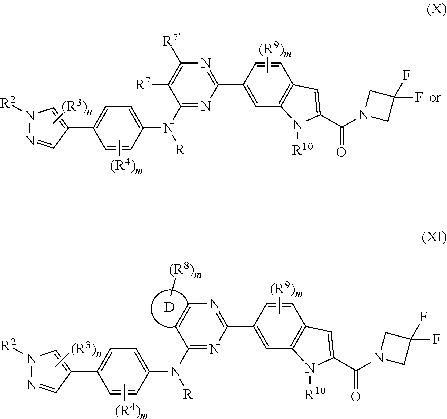

or a pharmaceutically acceptable salt, ester, stereoisomer, polymorph, solvate, N-oxide, or isotopically labeled compound thereof, wherein:
R is selected from the group consisting of H and C₁_s alkyl;
ring D is saturated or partially unsaturated 3- to 10-membered heterocycle, C₆₋₁₀ aryl or 5- to 10-membered heteroaromatic ring, preferably

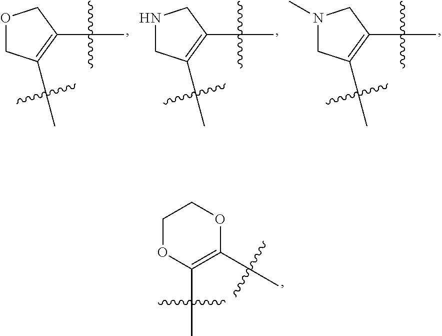

phenyl ring, N-methylpyrrole ring, furan ring or thiophene ring;
R² is selected from the group consisting of H and C₁₋₆ alkyl;
R³, R⁴, R⁷, R^{7′} and R⁸, at each occurrence, are each independently selected from the group consisting of H, halogen, —NH₂, —OH, C₁₋₆ alkyl and —OR⁵;
R⁹ and R¹⁰, at each occurrence, are each independently selected from the group consisting of H, halogen, C₁₋₆ alkyl, C₂₋₆ alkenyl, C₃₋₁₀ cyclic hydrocarbyl, 3- to 10-membered heterocyclyl, C₆₋₁₀ aryl, 5- to 14-membered heteroaryl, C₆₋₁₂ aralkyl, —C(═O)R⁵ and
—C₁₋₆ alkylene-O(P═O)(OH)₂;
the above alkyl, alkenyl, cyclic hydrocarbyl, heterocyclyl, aryl, heteroaryl, heteroaromatic ring and aralkyl, at each occurrence, are each optionally substituted with one or more substituents independently selected from the group consisting of halogen, C₁₋₆ alkyl and —OR⁵;
R⁵ and R⁶, at each occurrence, are each independently selected from the group consisting of H, C₁₋₆ alkyl, C₃₋₁₀ cyclic hydrocarbyl, 3- to 10-membered heterocyclyl, C₆₋₁₀ aryl, 5- to 14-membered heteroaryl and C₆₋₁₂ aralkyl;
m, at each occurrence, is each independently an integer of 0, 1, 2 or 3; and
n is an integer of 0, 1 or 2.
7. The method according to any one of embodiments 4 to 6, wherein R⁵ and R⁶, at each occurrence, are each independently selected from the group consisting of H, methyl and ethyl.
8. The method according to any one of embodiments 4 to 7, wherein R³, R⁴, R⁷, R^{7′} and R⁸, at each occurrence, are each independently selected from the group consisting of H, F, Cl, Br, —NH₂, —OH, methyl, trifluoromethyl, —CH₂-Ph, methoxy, ethoxy and —CH₂OCH₃.
9. The method according to any one of embodiments 4 to 8, wherein R⁹ and R¹⁰, at each occurrence, are each independently selected from the group consisting of H, F, Cl, Br, methyl, ethyl, n-propyl, isopropyl, vinyl, cyclopropyl, cyclobutyl, cyclopentyl, oxetanyl, monofluoromethyl, difluoromethyl, trifluoromethyl, acetyl, —OCH₂CHF₂, CH₂OH, —CH₂OCH₃, —CH₂CH₂OCH₃, —CH₂—O(P═O)(OH)₂,

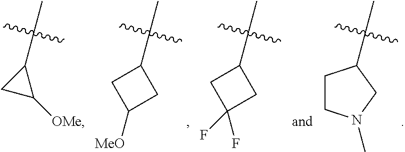

10. The method of any one of embodiments 1-3, wherein the selective inhibitor of ROCK2 is the compound (6-(4-((4-(1H-pyrazol-4-yl)phenyl)amino)pyrimidin-2-yl)-1-methyl-1H-indol-2-yl)(3,3-difluoroazetidin-1-yl)methanone having the chemical Formula XII:

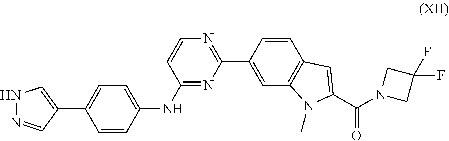 [SMILES: Cn1c(C(=O)N2CC(F)(F)C2)cc2ccc(-c3nccc(Nc4ccc(-c5cn[nH]c5)cc4)n3)cc21]

or a pharmaceutically acceptable salt, ester, stereoisomer, polymorph, solvate, N-oxide, or isotopically labeled compound thereof.
11. The method according to any one of embodiments 1-3, wherein the selective inhibitor of ROCK2 is a compound according to Formula XIII:

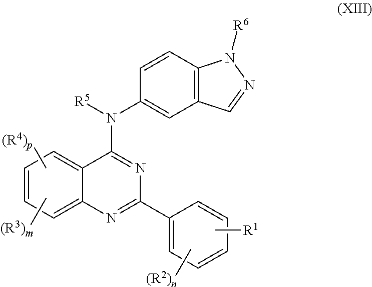

or a pharmaceutically acceptable salt, ester, stereoisomer, polymorph, solvate, N-oxide, or isotopically labeled compound thereof, wherein:
   R¹ is selected from the group consisting of —O—(CH₂)ᵣCO₂R¹², —O—(CH₂)_{y}, —C(═O)NR¹³R¹⁴, —O—(CH₂)_{y}, -heteroaryl, —O—(CH₂)_{y}, -cycloalkyl, —O—C(═O)—(CH₂)_{y}, —NR¹³R¹⁴, —O—(CH₂)_{z}, —NR¹³R¹⁴, —NH—C(═O) (CH₂)_{y}, —NR¹³R¹⁴, —NH—C(═O)—X—R¹⁵, and —NH—(CH₂)_{y}, —NR¹³R¹⁴;
   R¹² is selected from the group consisting of C₁-C₆ alkyl, —(C₁-C₆ alkyl)-O—(C₁-C₆alkyl), —(C₁-C₆ alkyl)-NR¹⁶R¹⁷, (C₁-C₆ alkyl)-C(═O)NR¹⁶R¹⁷, —(C₁-C₆ alkyl)-O—(C₁-C₆ alkyl)-O—(C₁-C₆ alkyl), aryl, aralkyl, heteroaryl, C₃-C₇ cycloalkyl, a three to twelve membered heterocyclic ring containing up to 3 heteroatoms, each of which may be optionally substituted at one or more carbon atoms by from 1 to 3 substituents independently selected from halo, C₁-C₆ alkoxy, hydroxy, amino, cyano and C₁-C₃ perfluoro alkyl;
   R¹³ and R¹⁴ are independently selected from the group consisting of H, C₁-C₈ alkyl, C₂-C₈ alkenyl, C₂-C₈ alkynyl, (C₁-C₆ alkyl)-O—(C₁-C₆ alkyl), —(C₁-C₆ alkyl)-NR¹⁶R¹⁷, —(C₁-C₆alkyl)-C(═O)NR¹⁶R¹⁷, aryl, aralkyl, heteroaryl, C₃-C₇ cycloalkyl, a three to twelve membered heterocyclic ring containing up to 3 heteroatoms, each of which may be optionally substituted by from 1 to 3 substituents independently selected from halo, C₁-C₆ alkyl, C₂-C₆ alkenyl, C₃-C₇ cycloalkyl. C₁-C₆ alkoxy, hydroxy, amino, cyano and C₁-C₃ perfluoro alkyl;
   or R¹³ and R¹⁴ may be taken together to form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆alkoxy, C₃-C₇ cycloalkyl, oxo, hydroxy, amino, cyano and C₁-C₃ perfluoro alkyl;
   X is absent or selected from a —O, NH, and C₁-C₆ alkyl;
   R¹⁵ is selected from the group consisting of heteroaryl, C₃-C₂ cycloalkyl, a three to twelve membered heterocyclic ring containing up to 3 heteroatoms, each of which may be optionally substituted by from 1 to 3 substituents independently selected from halo, C₁-C₆ alkyl, C₂-C₆ alkenyl. C₁-C₆ alkoxy, hydroxy, amino, cyano and C₁-C₃ perfluoro alkyl,
   or R¹⁵ is selected from —(C₁-C₆ alkyl)-O—(C₁-C₆ alkyl), —(C₁-C₆ alkyl)-NR¹⁶R¹⁷, —CO₂R¹⁸, —O—(CH₂)ₓ—CO₂R¹⁸, and C(═O)NR¹⁶R¹⁷;
   R¹⁶ and R¹⁷ are independently selected from the group consisting of H, C₁-C₈ alkyl, C₂-C₈ alkenyl, C₂-C₈ alkynyl, (C₁-C₆ alkyl)-O—(C₁-C₆ alkyl), aryl, aralkyl, heteroaryl, C₃-C₂ cycloalkyl, a three to twelve membered heterocyclic ring containing up to 3 heteroatoms, each of which may be optionally substituted by from 1 to 3 substituents independently selected from halo, C₁-C₆ alkyl, C₂-C₆ alkenyl, C₁-C₆ alkoxy, hydroxy, amino, cyano and C₁-C₃ perfluoro alkyl;
   or R¹⁶ and R¹⁷ may be taken together to form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆alkoxy, oxo, hydroxy, amino, cyano and C₁-C₃ perfluoro alkyl;
   R¹⁸ is selected from the group consisting of H, aryl, aralkyl, heteroaryl, C₁-C₆ alkyl, (C₁-C₆alkyl)-O—(C₁-C₆alkyl), —(C₁-C₆alkyl)-NR¹⁶R¹⁷, (C₁-C₆alkyl)-O—(C₁-C₆ alkyl)-O—(C₁-C₆ alkyl), each of which may be optionally substituted by from 1 to 3 substituents independently selected from halo, C₁-C₆ alkoxy, hydroxy, amino, cyano and C₁-C₃ perfluoroalkyl;
   x is selected from 0 to 6;
   y is selected from 0 to 6;
   z is selected from 2 to 6;
   each R² is independently selected from the group consisting of lower alkyl, CN, halo, hydroxy, lower alkoxy, amino, and perfluoro lower alkyl;
   each R³ is independently selected from the group consisting of lower alkyl, CN, halo, hydroxy, lower alkoxy, amino, and perfluoro lower alkyl;
   R⁴ is selected from H, —(CH₂)ₐ—NR⁴³R⁴⁴, —Y—R⁴², —O—(CH₂)ₐ—CO₂R⁴², —O—(CH₂)ₐ—C(═O)NR⁴³R⁴⁴, —O—(CH₂)ₐ-heteroaryl, —O—(CH₂)ₐ-cycloalkyl, —O—C(═O)—(CH₂)ₐ—NR⁴³R⁴⁴, —O—(CH₂)_{c}—N⁴³R⁴⁴, —NH—C(═O)—Y—R⁴⁵, —NH—C(═O)—(CH₂)ₐ—NR⁴³R⁴⁴;
   R⁴² is selected from the group consisting of C₁-C₆ alkyl, (C₁-C₆ alkyl)-O—(C₁-C₆alkyl), —(C₁-C₆ alkyl)-NR⁴⁶R⁴⁷, (C₁-C₆ alkyl)-C(═O)N⁴⁶R⁴⁷, (C₁-C₆ alkyl)-O—(C₁-C₆ alkyl)-O—(C₁-C₆ alkyl), each of which may be optionally substituted at one or more carbon atoms by from 1 to 3 substituents independently selected from halo, C₁-C₆ alkoxy, hydroxy, amino, cyano and C₁-C₃ perfluoro alkyl;
   R⁴³ and R⁴⁴ are independently selected from the group consisting of H, C₁-C₈ alkyl, C₂-C₈ alkenyl, C₂-C₈ alkynyl, (C₁-C₆ alkyl)-O—(C₁-C₆ alkyl), —(C₁-C₆ alkyl)-NR⁴⁶R⁴⁷, (C₁-C₆ alky)-C(═O)NR⁴⁶R⁴⁷, aryl, aralkyl, heteroaryl, C₃-C₇ cycloalkyl, a three to twelve membered heterocyclic ring containing up to 3 heteroatoms, each of which may be optionally substituted by from 1 to 3 substituents independently selected from halo, C₁-C₆ alkyl, C₂-C₆ alkenyl, C₃-C₇ cycloalkyl, C₁-C₆ alkoxy, hydroxy, amino, cyano and C₁-C₃ perfluoro alkyl;
   or R⁴³ and R⁴⁴ may be taken together to form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, oxo, hydroxy, amino, cyano and C₁-C₃ perfluoro alkyl;
   Y is absent or selected from O, NH, and C₁-C₆ alkyl;
   R⁴⁵ is selected from the group consisting of H, aryl, —(C₁-C₆ alkyl)-O—(C₁-C₆ alkyl), (C₁-C₆ alkyl)-NR⁴⁶R⁴⁷, —CO₂R⁴⁸, —O—(CH₂)₆—CO₂R⁴⁸, and —C(═O)NR⁴⁶R⁴⁷,
   R⁴⁶ and R⁴⁷ independently selected from the group consisting of H, C₁-C₈ alkyl, C₂-C₈ alkenyl, C₂-C₈ alkynyl, (C₁-C₆ alkyl)-O—(C₁-C₆ alkyl), aryl, aralkyl, heteroaryl, C₃-C₇ cycloalkyl, a three to twelve membered heterocyclic ring containing up to 3 heteroatoms, each of which may be optionally substituted by from 1 to 3 substituents independently selected from halo, C₁-C₆ alkyl, C₂-C₆ alkenyl, C₁-C₆ alkoxy, hydroxy, amino, cyano and C₁-C₃ perfluoro alkyl;
   or R⁴⁶ and R⁴⁷ may be taken together to form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, oxo, hydroxy, amino, cyano and C₁-C₃ perfluoro alkyl;
   R⁴⁸ is selected from the group consisting of H, aryl, aralkyl, heteroaryl, C₁-C₆ alkyl, (C₁-C₆ alkyl)-O—(C₁-C₆ alkyl), —(C₁-C₆ alkyl)-NR⁴⁶R⁴⁷, (C₁-C₆ alkyl)-O—(C₁-C₆ alkyl)-O—(C₁-C₆ alkyl), each of which may be optionally substituted by from 1 to 3 substituents independently selected from halo, C₁-C₆ alkoxy, hydroxy, amino, cyano and C₁-C₃ perfluoroalkyl;
   a is selected from 0 to 6;
   b is selected from 0 to 6;
   c is selected from 2 to 6;
   R⁵ is selected from the group consisting of H, C₁-C₆ alkyl, —(CH₂)_{d}—C(═O)—NR⁵³R⁵⁴, —C(═O)—(CH₂)_{d}—NR⁵³R⁵⁴, and —C(═O)—X—R⁵⁵;
   R⁵³ and R⁵⁴ are independently selected from the group consisting of H, C₁-C₈ alkyl, C₂-C₈ alkenyl, C₂-C₈ alkynyl, —(C₁-C₆ alkyl)-O—(C₁-C₆ alkyl), —(C₁-C₆ alkyl)-NR⁵⁶R⁵⁷, —(C₁-C₆ alkyl)-C(═O)NR⁵⁶R⁵⁷, aryl, aralkyl, heteroaryl, C₃-C₇ cycloalkyl, a three to twelve membered heterocyclic ring containing up to 3 heteroatoms, each of which may be optionally substituted by from 1 to 3 substituents independently selected from halo, C₁-C₆ alkyl, C₂-C₆ alkenyl, C₃-C₇ cycloalkyl, C₁-C₆ alkoxy, hydroxy, amino, cyano and C₁-C₃ perfluoro alkyl;
   or R⁵³ and R⁵⁴ may be taken together to form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, C₃-C₇ cycloalkyl, oxo, hydroxy, amino, cyano and C₁-C₃ perfluoro alkyl;
   R⁵⁵ is selected from the group consisting of H, aryl, —(C₁-C₆ alkyl)-O—(C₁-C₆ alkyl), (C₁-C₆ alkyl)-NR⁵⁶R⁵⁷, —CO₂R⁵⁸, (CH₂)_{c}—CO₂R⁵⁸, and —C(═O)NR⁵⁶R⁵⁷,
   R⁵⁶ and R⁵⁷ independently selected from the group consisting of H, C₁-C₈ alkyl, C₂-C₈ alkenyl, C₂-C₈ alkynyl, —(C₁-C₆ alkyl)-O—(C₁-C₆ alkyl), aryl, aralkyl, heteroaryl, C₃-C₇ cycloalkyl, a three to twelve membered heterocyclic ring containing up to 3 heteroatoms, each of which may be optionally substituted by from 1 to 3 substituents independently selected from halo, C₁-C₆ alkyl, C₂-C₆ alkenyl, C₁-C₆ alkoxy, hydroxy, amino, cyano and C₁-C₃ perfluoro alkyl;
   or R⁵⁶ and R⁵⁷ may be taken together to form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, oxo, hydroxy, amino, cyano and C₁-C₃ perfluoro alkyl;
   R⁵⁸ is selected from the group consisting of H, aryl, aralkyl, heteroaryl, C₁-C₆ alkyl, (C₁-C₆ alkyl)-O—(C₁-C₆ alkyl), —(C₁-C₆ alkyl)-NR⁵⁶R⁵⁷, (C₁-C₆ alkyl)-O—(C₁-C₆alkyl)-O—(C₁-C₆ alkyl), each of which may be optionally substituted by from 1 to 3 substituents independently selected from halo, C₁-C₆ alkoxy, hydroxy, amino, cyano and C₁-C₃ perfluoroalkyl;
   d is selected from 0 to 6;
   e is selected from 0 to 6;
   R⁶ is selected from the group consisting of H, C₁-C₆ alkyl, —(CH₂)ᵣ—C(═O)NR⁶³R⁶⁴, —C(═O)—(CH₂)ᵣ—NR⁶³R⁶⁴, and C(═O)—X—R⁶⁵;
   R⁶³ and R⁶⁴ are independently selected from the group consisting of H, C₁-C₈ alkyl, C₂-C₈ alkenyl, C₂-C₈ alkynyl, —(C₁-C₆ alkyl)-O—(C₁-C₆ alkyl), —(C₁-C₆ alkyl)-NR⁶⁶R⁶⁷, (C₁-C₆ alkyl)-C(═O)NR⁶⁶R⁶⁷, aryl, aralkyl, heteroaryl, C₃-C₇ cycloalkyl, a three to twelve membered heterocyclic ring containing up to 3 heteroatoms, each of which may be optionally substituted by from 1 to 3 substituents independently selected from halo, C₁-C₆ alkyl, C₂-C₆ alkenyl. C₃-C₇ cycloalkyl, C₁-C₆ alkoxy, hydroxy, amino, cyano and C₁-C₃ perfluoro alkyl;
   or R⁶³ and R⁶⁴ may be taken together to form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆alkoxy, C₃-C₇cycloalkyl, oxo, hydroxy, amino, cyano and C₁-C₃ perfluoro alkyl;
   R⁶⁵ is selected from the group consisting of H, aryl, —(C₁-C₆ alkyl)-O—(C₁-C₆ alkyl), (C₁-C₆ alkyl)-NR⁶⁶R⁶⁷, —CO₂R, —O—(CH₂), —CO₂R⁶⁸, and —C(═O)NR⁶⁶R⁶⁷,
   R⁶⁶ and R⁶⁷ independently selected from the group consisting of H, C₁-C₅ alkyl, C₂-C₈ alkenyl, C₂-C₈ alkynyl, —(C₁-C₆ alkyl)-O—(C₁-C₆ alkyl), aryl, aralkyl, heteroaryl, C₃-C₇ cycloalkyl, a three to twelve membered heterocyclic ring containing up to 3 heteroatoms, each of which may be optionally substituted by from 1 to 3 substituents independently selected from halo, C₁-C₆ alkyl, C₂-C₆ alkenyl, C₁-C₆ alkoxy, hydroxy, amino, cyano and C₁-C₃ perfluoro alkyl;
   or R⁶⁶ and R⁶⁷ may be taken together to form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, oxo, hydroxy, amino, cyano and C₁-C₃ perfluoro alkyl;
   R⁶⁸ is selected from the group consisting of H, aryl, aralkyl, heteroaryl, C₁-C₆ alkyl, —(C₁-C₆ alkyl)-O—(C₁-C₆ alkyl), —(C₁-C₆ alkyl)-NR⁶⁶R⁶⁷, —(C₁-C₆ alkyl)-O—(C₁-C₆alkyl)-O—(C₁-C₆ alkyl), each of which may be optionally substituted by from 1 to 3 substituents independently selected from halo, C₁-C₆ alkoxy, hydroxy, amino, cyano and C₁-C₃ perfluoroalkyl;
   r is selected from 0 to 6;
   s is selected from 0 to 6;
   n is selected from 0 to 4;
   m is selected from 0 to 3; and
   p is selected from 0 and 1.
12. The method of embodiment 11, wherein the selective inhibitor of ROCK2 is a compound according to Formula XIV:

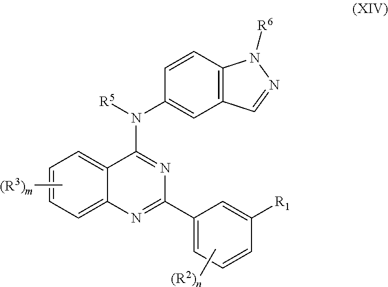

or a pharmaceutically acceptable salt, ester, stereoisomer, polymorph, solvate, N-oxide, or isotopically labeled compound thereof, wherein R¹, R², R³, R⁵, R⁶, m and n are as defined in Formula XIII.
13. The method of embodiment 11 or 12, wherein the selective inhibitor of ROCK2 is a compound according to Formula XV:

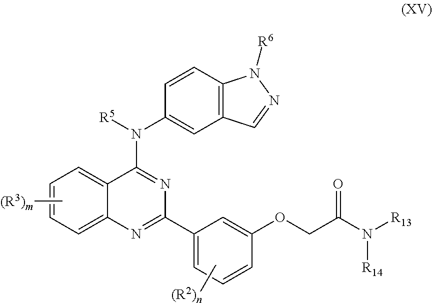

or a pharmaceutically acceptable salt, ester, stereoisomer, polymorph, solvate, N-oxide, or isotopically labeled compound thereof, wherein
R¹³ and R¹⁴ are independently selected from the group consisting of H, C₁-C₈ alkyl, C₂-C₈ alkenyl, C₂-C₈ alkynyl, (C₁-C₆ alkyl)-O—(C₁-C₆ alkyl), —(C₁-C₆ alkyl)-NR¹⁶R¹⁷, —(C₁-C₆ alkyl)-C(═O)NR¹⁶R¹⁷, aryl, aralkyl, heteroaryl, C₃-C₇ cycloalkyl, a three to twelve membered heterocyclic ring containing up to 3 heteroatoms, each of which may be optionally substituted by from 1 to 3 substituents independently selected from halo, C₁-C₆ alkyl, C₂-C₆ alkenyl, C₃-C₇ cycloalkyl, C₁-C₆ alkoxy, hydroxy, amino, cyano and C₁-C₃ perfluoro alkyl;
or R¹³ and R¹⁴ may be taken together to form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆alkoxy, C₃-C₇ cycloalkyl, oxo, hydroxy, amino, cyano and C₁-C₃ perfluoro alkyl;
R¹⁶ and R¹⁷ are independently selected from the group consisting of H, C₁-C₈ alkyl, C₂-C₈ alkenyl, C₂-C₈ alkynyl, —(C₁-C₆ alkyl)-O—(C₁-C₆ alkyl), aryl, aralkyl, heteroaryl, C₃-C₇ cycloalkyl, a three to twelve membered heterocyclic ring containing up to 3 heteroatoms, each of which may be optionally substituted by from 1 to 3 substituents independently selected from halo, C₁-C₆ alkyl, C₂-C₆ alkenyl, C₁-C₆alkoxy, hydroxy, amino, cyano and C₁-C₃ perfluoro alkyl;
or R¹ and R¹⁷ may be taken together to form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆alkoxy, oxo, hydroxy, amino, cyano and C₁-C₃ perfluoro alkyl; and
wherein each of R², R³, R⁵, R⁶, m and n are as defined in Formula XIII.
14. The method of embodiment 13, wherein the selective inhibitor of ROCK2 is a compound according to Formula XVI:

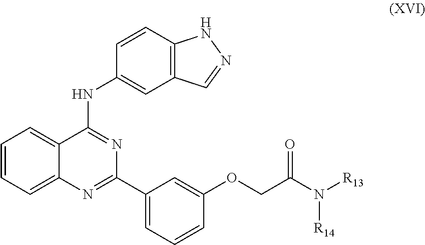

or a pharmaceutically acceptable salt, ester, stereoisomer, polymorph, solvate, N-oxide, or isotopically labeled compound thereof, wherein R¹³ and R¹⁴ are as defined in Formula XV.
15. The method of embodiment 14, wherein the selective inhibitor of ROCK2 is (2-(3-(4-((1H-indazol-5-yl)amino)quinazolin-2-yl)phenoxy)-N-isopropylacetamide) having the chemical Formula XVII:

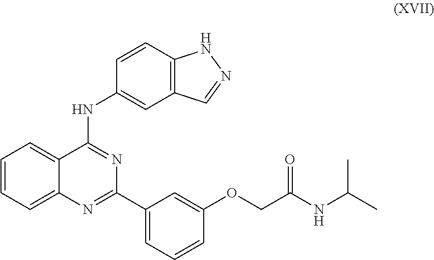 [SMILES: CC(C)NC(=O)COc1cccc(-c2nc(Nc3ccc4[nH]ncc4c3)c3ccccc3n2)c1]

or a pharmaceutically acceptable salt, ester, stereoisomer, polymorph, solvate, N-oxide, or isotopically labeled compound thereof.
16. The method of any one of embodiments 1-3, wherein the selective inhibitor of ROCK2 is a ribonucleic acid (RNA).
17. The method of embodiment 16, wherein the RNA is an antisense RNA against ROCK2 transcription.
18. The method of embodiment 16, wherein the RNA is a small interfering RNA (siRNA) or micro RNA (miRNA).
19. The method of any one of embodiments 1-18, wherein the subject is a human.
20. The method of any one of embodiments 1-19, wherein the subject has previous been administered an additional muscular dystrophy treatment.
21. The method of any one of embodiments 1-20, wherein the subject is concurrently being administered an additional muscular dystrophy treatment.
22. The method of any one of embodiments 1-21, wherein the subject will subsequently be administered an additional muscular dystrophy treatment.
23. The method of any one of embodiments 20-22, wherein the additional muscular dystrophy treatment is selected from the group consisting of a corticosteroid, an angiotensin-converting enzyme (ACE) inhibitor, a beta blocker, an exon skipping oligonucleotide, and a gene therapy.
24. The method of embodiment 23, wherein the corticosteroid is prednisone or deflazacort.
25. The method of embodiment 23, wherein the exon skipping oligonucleotide is eteplirsen or golodirsen.
26. The method of embodiment 23, wherein the gene therapy is SRP-9001 or PF-06939926.

### EXAMPLES

### Example 1: In Vitro Evaluation of Selective Inhibitors of ROCK2 for Treating Duchenne Muscular Dystrophy

In vitro methods of evaluating the effect of RhoA pathway inhibitors on Duchenne muscular dystrophy (DMD) are known in the art. See, e.g., Fernández-Simón, et al., J. Cachexia, 2022, vol. 13:1373-1384.

Skeletal muscle fibro-adipogenic cells (FAPs) will be obtained from muscle biopsies from DMD patients. FAPs will be cultured in the presence of vehicle or a selective inhibitor of ROCK2 disclosed herein (e.g., Formula XII or Formula XVII) or a pharmaceutically acceptable salt, ester, stereoisomer, polymorph, solvate, N-oxide, or isotopically labeled compound thereof. Subsequently, a platelet-derived growth factor (PDGF) will be added to the culture. The cells will then be evaluated for cell viability, RhoA-GTP activity (e.g., G-LISA assay), ROCK2 activity, filamentous actin content, soluble collagen release, cytokine production, proliferation, migration, and by Western blot analysis. It is expected that the cells cultured in the presence of a selective inhibitor of ROCK2 will demonstrate decreased PDGF-mediated proliferation, migration, and collagen release.

### Example 2: In Vivo Evaluation of Selective Inhibitors of ROCK2 for Treating Duchenne Muscular Dystrophy

Several acceptable animal models of Duchenne muscular dystrophy (DMD) are known in the art. See, e.g., McGreevy et al., Dis. Model Mech., 2015, vol. 8(3): 195-213.

Male DMD model animals (e.g., mdx mice, such as DBA/2-mdx mice) will be administered either vehicle or a selective inhibitor of ROCK2 disclosed herein (e.g., Formula XII or Formula XVII) or a pharmaceutically acceptable salt, ester, stereoisomer, polymorph, solvate, N-oxide, or isotopically labeled compound thereof. Male wildtype animals (e.g., male DBA/2 mice) will be used as healthy controls. Following treatment, animals will be evaluated for muscle function (e.g., using a grip-strength test, a hanging test, rotarod running, and/or forced treadmill running). See, e.g., Aartsma-Rus and van Putten, J. Vis. Exp., 2014, vol. 85: e51303.

The animals will also be evaluated for histology. For example, hematoxylin and eosin staining can be used to evaluate fibrotic lesions, mononuclear cell infiltration, and loss of muscle fibers. Immunohistochemistry may also be used to evaluate, e.g., collagen production and macrophage infiltration. Skeletal muscle (e.g., diaphragm, tongue, and quadriceps) and cardiac muscle may be histologically evaluated.

1. A method for treating muscular dystrophy in a subject in need thereof, the method comprising administering to the subject a therapeutically effective amount of a selective inhibitor of Rho-associated coiled-coil kinase 2 (ROCK2).

2. The method of claim 1, wherein the muscular dystrophy is selected from the group consisting of Duchenne muscular dystrophy, Becker muscular dystrophy, facioscapulohumeral muscular dystrophy, limb-girdle muscular dystrophy, myotonic muscular dystrophy, oculopharyngeal muscular dystrophy, tibial muscular dystrophy, congenital muscular dystrophy, distal muscular dystrophy, and Emery-Dreifuss muscular dystrophy.

3. The method of claim 2, wherein the muscular dystrophy is Duchenne muscular dystrophy.

4. The method of claim 1, wherein the selective inhibitor of ROCK2 is compound according to Formula I: 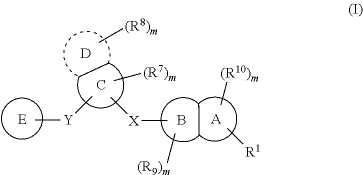
or a pharmaceutically acceptable salt, ester, stereoisomer, polymorph, solvate, N-oxide, or isotopically labeled compound thereof, wherein:
X and Y are each independently selected from the group consisting of a direct bond, C(═O), O, S(═O), and NR;
R is selected from the group consisting of H, C₁₋₆ alkyl, C₂₋₆ alkenyl, C₂₋₆ alkynyl, saturated or partially unsaturated C₃₋₁₀ cyclic hydrocarbyl, saturated or partially unsaturated 3- to 10-membered heterocyclyl, C₆₋₁₀ aryl, 5- to 14-membered heteroaryl and C₆₋₁₂ aralkyl, and at most 2 ring members in the cyclic hydrocarbyl and heterocyclyl are C(═O);
ring A and ring B are each independently selected from the group consisting of saturated or partially unsaturated C₃₋₁₀ hydrocarbon ring, saturated or partially unsaturated 3- to 10-membered heterocycle, C₆₋₁₀ aromatic ring and 5- to 14-membered heteroaromatic ring, and at most 2 ring members in the hydrocarbon ring and heterocycle are C(═O); provided that when ring B is a heterocycle containing a nitrogen atom, ring B is not attached to X via the nitrogen atom;
ring C is selected from the group consisting of saturated or partially unsaturated C₃₋₁₀ hydrocarbon ring, saturated or partially unsaturated 3- to 10-membered heterocycle, C₆₋₁₀ aromatic ring and 5- to 14-membered heteroaromatic ring, and at most 2 ring members in the hydrocarbon ring and heterocycle are C(═O);
ring D is absent, or is selected from the group consisting of saturated or partially unsaturated C₃₋₁₀ hydrocarbon ring, saturated or partially unsaturated 3- to 10-membered heterocycle, C₆₋₁₀ aromatic ring and 5- to 14-membered heteroaromatic ring, and at most 2 ring members in the hydrocarbon ring and heterocycle are C(═O);
ring E is selected from the group consisting of: 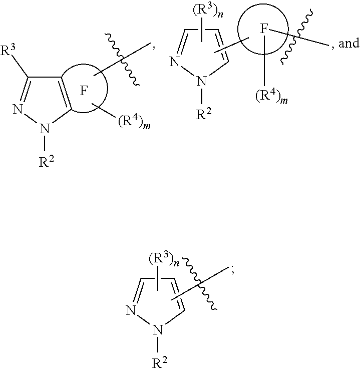
ring F is selected from the group consisting of saturated or partially unsaturated C₃₋₁₀ hydrocarbon ring, saturated or partially unsaturated 3- to 10-membered heterocycle, C₆₋₁₀ aromatic ring and 5- to 14-membered heteroaromatic ring, and at most 2 ring members in the hydrocarbon ring and heterocycle are C(═O);
R¹ is selected from the group consisting of H, —NH₂, C₁₋₆ alkyl, C₆₋₁₀ aryl, 5- to 14-membered heteroaryl, N-methylpyrrolidinyl, N-methylpiperidinyl, 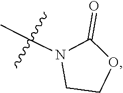 [SMILES: *N1CCOC1=O]
acetyl, 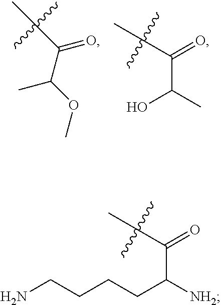
—C(═O)—(C₁₋₆ alkylene)n-CF₃, —C(═O)—(C₁₋₆ alkylene) CN, —C(═O)-(saturated or partially unsaturated C₃₋₁₀ cyclic hydrocarbyl), —NHC(═O)-(saturated or partially unsaturated C₃₋₁₀ cyclic hydrocarbyl), —C(═O)-(saturated or partially unsaturated 3- to 10-membered heterocyclyl), —C(═O)—C₁₋₆ alkylene-(saturated or partially unsaturated 3- to 10-membered heterocyclyl), —C(═O)-(5- to 14-membered heteroaryl), —C(═O)—C₁₋₆ alkylene-NH(C₁₋₆ alkyl), —C(═O)—C₁₋₆ alkylene-N(C₁₋₆ alkyl)₂, N-methylpiperazine substituted acetyl, —S(═O)₂R^{1a}, —P(═O)R^{1a}R^{1b}, 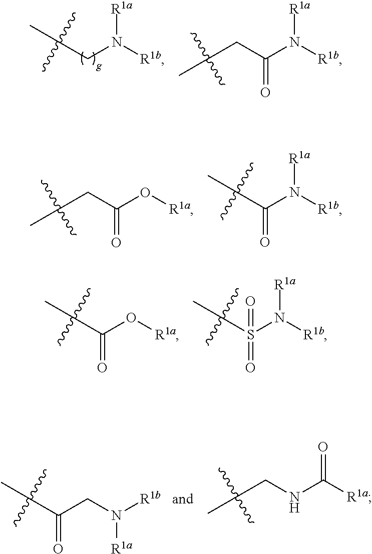
provided that when one of R¹ and R¹⁰ is C¹⁻⁶ alkyl, and the other is H or C³⁻¹⁰ cyclic hydrocarbyl, at least one of X and Y is a direct bond, and ring C is not a 5-membered heteroaromatic ring; when one of R¹ and R¹⁰ is H, and the other is 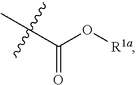
ring C is not a 5-membered heteroaromatic ring; when both R¹ and R¹⁰ are H, ring A contains at least one nitrogen atom, and is not a 5- or 6-membered ring; when one of R¹ and R¹⁰ is H, and the other is 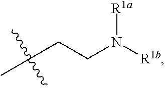
ring C is not a 5-membered heteroaromatic ring; and when one of R¹ and R¹⁰ is H, and the other is H or acetyl, ring D is absent;
R^{1a} and R^{1b} are each independently selected from the group consisting of H, halogen, amino, cyano, nitro, C₁₋₆ alkyl, C₂₋₆ alkenyl, C₂₋₆ alkynyl, C₃₋₁₀ cyclic hydrocarbyl, 3- to 10-membered heterocyclyl, C₆₋₁₀ aryl, 5- to 14-membered heteroaryl, C₆₋₁₂ aralkyl,
—C(═O)R⁵, —OC(═O)R⁵, —C(═O)OR⁵, —OR⁵, —SR⁵, —S(═O)R⁵, —S(═O)₂R⁵,
—S(═O)₂NR⁵R⁶, —NR⁵R⁶, —C(═O)NR⁵R⁶, —NR⁵—C(═O)R⁶, —NR⁵—C(═O)OR⁶,
NR⁵—S(═O)₂—R⁶, —NR⁵—C(═O)—NR⁵R⁶, —C₁₋₆ alkylene-NR⁵R⁶, —C₁₋₆ alkylene-OR⁵ and
—O—C₁₋₆ alkylene-NR⁵R⁶, provided that when one of R^{1a} and R^{1b} is n-propyl, the other is not H; or R^{1a} and R^{1b} together with the atom to which they are attached form a 3- to 12-membered heterocycle or heteroaromatic ring;
R², R³, R⁴, R⁷, R⁸, R⁹ and R¹⁰, at each occurrence, are each independently selected from the group consisting of H, halogen, amino, cyano, nitro, C₁₋₆ alkyl, C₂₋₆ alkenyl, C₂₋₆ alkynyl, C₃₋₁₀ cyclic hydrocarbyl, 3- to 10-membered heterocyclyl, C₆₋₁₀ aryl, 5- to 14-membered heteroaryl, C₆₋₁₂ aralkyl, —C(═O)R⁵, OC(═O)R⁵, —C(═O)OR⁵, OR⁵, SR⁵, —S(═O)R⁵, —S(═O)₂R⁵, —S(═O)₂NR⁵R⁶, —NR⁵R⁶, —C(═O)NR⁵R⁶, —NR⁵—C(═O)R⁶,
—NR⁵—C(═O)OR⁶, —NR⁵—S(═O)₂—R⁶, —NR⁵—C(═O)—NR⁵R⁶, —C₁₋₆ alkylene-NR⁵R⁶,
—C₁₋₆ alkylene-O(P═O)(OH)₂ and —O—C₁₋₆ alkylene-NR⁵R⁶;
the above alkyl, alkylene, alkenyl, alkynyl, cyclic hydrocarbyl, hydrocarbon ring, heterocyclyl, heterocycle, aryl, aromatic ring, heteroaryl, heteroaromatic ring and aralkyl, at each occurrence, are each optionally substituted with one or more substituents independently selected from the group consisting of halogen, hydroxyl, oxo, amino, cyano, nitro, C₁₋₆ alkyl, C₂₋₆ alkenyl, C₂₋₆ alkynyl, C₃₋₆ cyclic hydrocarbyl, 3- to 10-membered heterocyclyl, C₆₋₁₀ aryl, 5- to 14-membered heteroaryl,
C₆₋₁₂ aralkyl, ═N—OR⁵, —C(═NH)NH₂, —C(═O)R⁵, —OC(═O)R⁵, —C(═O)OR⁵, —OR⁵,
—SR⁵, —S(═O)R⁵, —S(═O)₂R⁵, —S(═O)₂NR⁵R⁶, —NR⁵R⁶, —C(═O)NR⁵R⁶, —NR⁵—C(═O)R⁶, —NR⁵—C(═O)OR⁶, —NR⁵—S(═O)₂—R⁶, —NR⁵—C(═O)—NR⁵R⁶, —C₁₋₆ alkylene-NR⁵R⁶ and
—O—C₁₋₆ alkylene-NR⁵R⁶, and the alkyl, cyclic hydrocarbyl, heterocyclyl, aryl, heteroaryl and aralkyl are further optionally substituted with one or more substituents independently selected from the group consisting of halogen, hydroxyl, oxo, amino, cyano, nitro, C₁₋₆ alkyl, C₃₋₆ cyclic hydrocarbyl, 3- to 10-membered heterocyclyl, C₆₋₁₀ aryl, 5- to 14-membered heteroaryl and C₆₋₁₂ aralkyl;
R⁵ and R⁶, at each occurrence, are each independently selected from the group consisting of H, alkyl, C₃₋₁₀ cyclic hydrocarbyl, 3- to 10-membered heterocyclyl, C₆₋₁₀ aryl, 5- to 14-membered heteroaryl and C₆₋₁₂ aralkyl;
m, at each occurrence, is each independently an integer of 0, 1, 2 or 3;
n is an integer of 0, 1 or 2;
i is an integer of 0, 1 or 2; and
g is an integer of 0, 1, 2, 3 or 4.

5. The method of claim 4, wherein the selective inhibitor of ROCK2 is a compound according to any one of Formula II to IX: 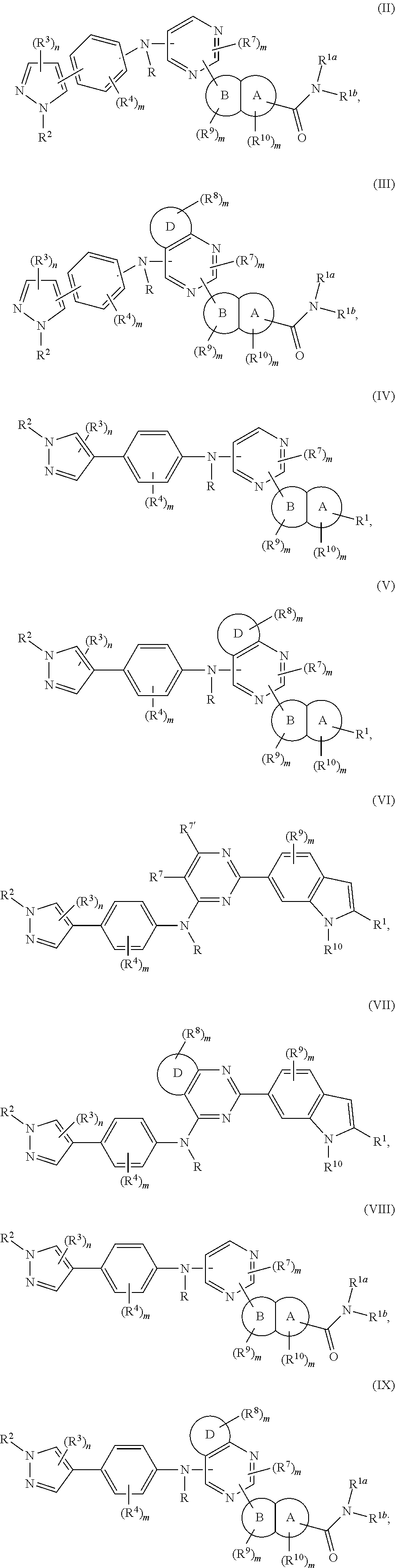
or a pharmaceutically acceptable salt, ester, stereoisomer, polymorph, solvate, N-oxide, or isotopically labeled compound thereof, wherein each of ring A, ring B, ring D, R, R¹, R^{1a}, R^{1b}, R², R³, R⁴, R⁷, R^{7′}, R⁸, R⁹, R¹⁰, n and m are defined for Formula I.

6. The method of claim 5, wherein the selective inhibitor ROCK2 is a compound according to Formula X or formula XI: 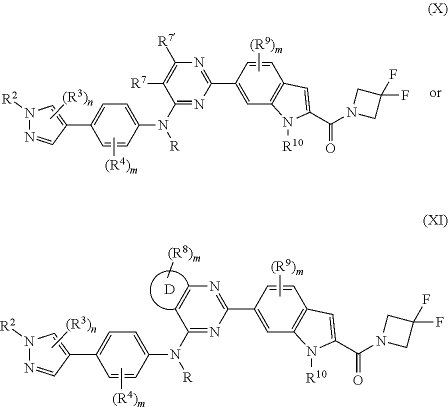
or a pharmaceutically acceptable salt, ester, stereoisomer, polymorph, solvate, N-oxide, or isotopically labeled compound thereof, wherein:
R is selected from the group consisting of H and C₁₋₆ alkyl;
ring D is saturated or partially unsaturated 3- to 10-membered heterocycle, C₆₋₁₀ aryl or 5- to 10-membered heteroaromatic ring, preferably 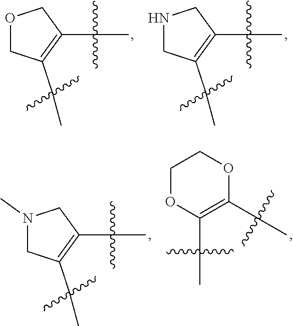
phenyl ring, N-methylpyrrole ring, furan ring or thiophene ring;
R² is selected from the group consisting of H and C₁₋₆ alkyl;
R³, R⁴, R⁷, R^{7′} and R⁸, at each occurrence, are each independently selected from the group consisting of H, halogen, —NH₂, —OH, C₁₋₆ alkyl and —OR⁵;
R⁹ and R¹⁰, at each occurrence, are each independently selected from the group consisting of H, halogen, C¹⁻⁶ alkyl, C₂₋₆ alkenyl, C₃₋₁₀ cyclic hydrocarbyl, 3- to 10-membered heterocyclyl, C₆₋₁₀ aryl, 5- to 14-membered heteroaryl, C₆₋₁₂ aralkyl, —C(═O)R⁵ and
—C₁₋₆ alkylene-O(P═O)(OH)₂;
the above alkyl, alkenyl, cyclic hydrocarbyl, heterocyclyl, aryl, heteroaryl, heteroaromatic ring and aralkyl, at each occurrence, are each optionally substituted with one or more substituents independently selected from the group consisting of halogen, C₁₋₆ alkyl and —OR⁵;
R⁵ and R⁶, at each occurrence, are each independently selected from the group consisting of H, C₁₋₆ alkyl, C₃₋₁₀ cyclic hydrocarbyl, 3- to 10-membered heterocyclyl, C₆₋₁₀ aryl, 5- to 14-membered heteroaryl and C₆₋₁₂ aralkyl;
m, at each occurrence, is each independently an integer of 0, 1, 2 or 3; and
n is an integer of 0, 1 or 2.

7. The method of claim 4, wherein R⁵ and R⁶, at each occurrence, are each independently selected from the group consisting of H, methyl and ethyl.

8. The method of claim 4, wherein R³, R⁴, R⁷, R^{7′} and R⁸, at each occurrence, are each independently selected from the group consisting of H, F, Cl, Br, —NH₂, —OH, methyl, trifluoromethyl, —CH₂-Ph, methoxy, ethoxy and —CH₂OCH₃.

9. The method of claim 4, wherein R⁹ and R¹⁰, at each occurrence, are each independently selected from the group consisting of H, F, Cl, Br, methyl, ethyl, n-propyl, isopropyl, vinyl, cyclopropyl, cyclobutyl, cyclopentyl, oxetanyl, monofluoromethyl, difluoromethyl, trifluoromethyl, acetyl, —OCH₂CHF₂, CH₂OH, —CH₂OCH₃, —CH₂CH₂OCH₃, —CH₂—O(P═O)(OH)₂,

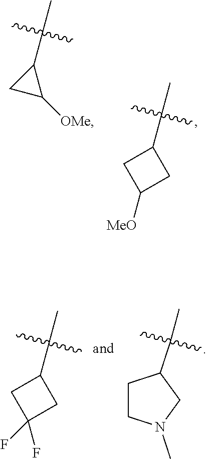

10. The method of claim 1, wherein the selective inhibitor of ROCK2 is the compound (6-(4-((4-(1H-pyrazol-4-yl)phenyl)amino)pyrimidin-2-yl)-1-methyl-1H-indol-2-yl)(3,3-difluoroazetidin-1-yl)methanone having the chemical Formula XII: 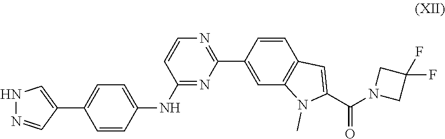 [SMILES: Cn1c(C(=O)N2CC(F)(F)C2)cc2ccc(-c3nccc(Nc4ccc(-c5cn[nH]c5)cc4)n3)cc21]
or a pharmaceutically acceptable salt, ester, stereoisomer, polymorph, solvate, N-oxide, or isotopically labeled compound thereof.

11. The method of claim 1, wherein the selective inhibitor of ROCK2 is a compound according to Formula XIII: 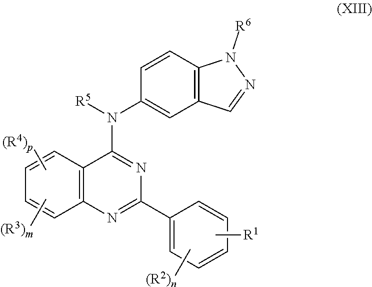
or a pharmaceutically acceptable salt, ester, stereoisomer, polymorph, solvate, N-oxide, or isotopically labeled compound thereof, wherein:
R¹ is selected from the group consisting of —O—(CH₂)ᵣCO₂R¹², —O—(CH₂)_{y}, —C(═O)NR¹³R¹⁴, —O—(CH₂)_{y}, -heteroaryl, —O—(CH₂)_{y}, -cycloalkyl, —O—C(═O)—(CH₂)_{y}, —NR¹³R¹⁴, —O—(CH₂)_{z}—NR¹³R¹⁴, —NH—C(═O)—(CH₂)_{y}, —NR¹³R¹⁴, —NH—C(═O)—X—R¹⁵, and —NH—(CH₂)_{y}, —NR¹³R¹⁴;
R¹² is selected from the group consisting of C₁-C₆ alkyl, —(C₁-C₆ alkyl)-O—(C₁-C₆ alkyl), —(C₁-C₆ alkyl)-NR¹⁶R¹⁷, —(C₁-C₆ alkyl)-C(═O)NR¹⁶R¹⁷, —(C₁-C₆, alky)-O—(C₁-C₆ alkyl)-O—(C₁-C₆ alkyl), aryl, aralkyl, heteroaryl, C₃-C₇ cycloalkyl, a three to twelve membered heterocyclic ring containing up to 3 heteroatoms, each of which may be optionally substituted at one or more carbon atoms by from 1 to 3 substituents independently selected from halo, C₁-C₆ alkoxy, hydroxy, amino, cyano and (C₁-C₆ perfluoro alkyl;
R¹³ and R¹⁴ are independently selected from the group consisting of H, C₁-C₈ alkyl, C₂-C₈ alkenyl, C₂-C₈ alkynyl, —C₁-C₆ alkyl)-O—(C₁-C₆ alkyl), —(C₁-C₆ alkyl)-NR¹⁶R¹⁷, —(C₁-C₆ alkyl)-C(═O)NR¹⁶R¹⁷, aryl, aralkyl, heteroaryl, C₃-C₇ cycloalkyl, a three to twelve membered heterocyclic ring containing tip to 3 heteroatoms, each of which may be optionally substituted by from 1 to 3 substituents independently selected from halo, C₁-C₆ alkyl, C₂-C₆ alkenyl, C₃-C₇ cycloalkyl, C₁-C₆ alkoxy, hydroxy, amino, cyano and C₁-C₃; perfluoro alkyl;
or R¹³ and R¹⁴ may be taken together to form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, C₃-C₇ cycloalkyl, oxo, hydroxy, amino, cyano and C₁-C₃, perfluoro alkyl;
X is absent or selected from a —O, NH, and C₁-C₆ alkyl;
R¹⁵ is selected from the group consisting of heteroaryl, C₃-C₇ cycloalkyl, a three to twelve membered heterocyclic ring containing up to 3 heteroatoms, each of which may be optionally substituted by from 1 to 3 substituents independently selected from halo, C₁-C₆ alkyl, C₂-C₆ alkenyl, C₁-C₆ alkoxy, hydroxy, amino, cyano and C₁-C₃; perfluoro alkyl,
or R¹⁵ is selected from —(C₁-C₆ alkyl)-O—(C₁-C₆ alkyl), —(C₁-C₆ alkyl)-NR¹⁶R¹⁷, —CO₂R¹⁸, —O—(CH₂)ₓ—CO₂R¹⁸, and C(═O)NR¹⁶R¹⁷;
R¹⁶ and R¹⁷ are independently selected from the group consisting of H, C₁-C₈ alkyl, C₂-C₈ alkenyl, C₂-C₈ alkynyl, —(C₁-C₆ alkyl)-O—C₁-C₆ alkyl), aryl, aralkyl, heteroaryl, C₃-C₇ cycloalkyl, a three to twelve membered heterocyclic ring containing up to 3 heteroatoms, each of which may be optionally substituted by from 1 to 3 substituents independently selected from halo, C₁-C₆, alkyl, C₂-C₆ alkenyl, C₁-C₆ alkoxy, hydroxy, amino, cyano and C₁-C₃ perfluoro alkyl;
or R¹⁶ and R¹⁷ may be taken together to form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, oxo, hydroxy, amino, cyano and C₁-C₃ perfluoro alkyl;
R¹⁸ is selected from the group consisting of H, aryl, aralkyl, heteroaryl, C₁-C₆ alkyl, —(C₁-C₆ alkyl)-O—(C₁-C₆ alkyl), —(C₁-C₆ alkyl)-NR¹⁶R¹⁷, —(C₁-C₆ alkyl)-O—(C₁-C₆ alkyl)-O—(C₁-C₆ alkyl), each of which may be optionally substituted by from 1 to 3 substituents independently selected from halo, C₁-C₆ alkoxy, hydroxy, amino, cyano and C₁-C₃ perfluoroalkyl;
x is selected from 0 to 6;
y is selected from 0 to 6;
z is selected from 2 to 6;
each R² is independently selected from the group consisting of lower alkyl, CN, halo, hydroxy, lower alkoxy, amino, and perfluoro lower alkyl;
each R³ is independently selected from the group consisting of lower alkyl, CN, halo, hydroxy, lower alkoxy, amino, and perfluoro lower alkyl;
R⁴ is selected from H, —(CH₂)ₐ—NR⁴³R⁴⁴, —Y—R⁴², —O—(CH₂)ₐ—CO₂R⁴², —O—(CH₂)ₐ—C(═O)NR⁴³R⁴⁴, —O— (CH₂)ₐ-heteroaryl, —O—(CH₂)ₐ-cycloalkyl, —O—C(═O)—(CH₂)ₐ—NR⁴³R⁴⁴, —O—(CH₂)_{c}—N⁴³R⁴⁴, —NH—C(═O)—Y—R⁴⁵, —NH—C(═O)—(CH₂)ₙ—NR⁴³R⁴⁴;
R⁴² is selected from the group consisting of C₁-C₆ alkyl, —(C₁-C₆ alkyl)-O—(C₁-C₆ alkyl, —(C₁-C₆ alkyl)-NR⁴⁶R⁴⁷, —(C₁-C₆ alkyl)-C(═O) N⁴⁶R⁴⁷, —(C₁-C₆ alkyl)-O—(C₁-C₆ alky)-O—(C₁-C₆ alkyl), each of which may be optionally substituted at one or more carbon atoms by from 1 to 3 substituents independently selected from halo, C₁-C₆ alkoxy, hydroxy, amino, cyano and C₁-C₃ perfluoro alkyl;
R⁴³ and R⁴⁴ are independently selected from the group consisting of H, C₁-C₈ alkyl, C₂-C₈ alkenyl, C₂-C₈ alkynyl, —(C₁-C₆ alkyl)-O—(C₁-C₆ alkyl), —(C₁-C₆ alkyl)-NR⁴⁶R⁴⁷, —(C₁-C₆ alkyl)-C(═O)NR⁴⁶R⁴⁷, aryl, aralkyl, heteroaryl, C₃-C₇ cycloalkyl, a three to twelve membered heterocyclic ring containing up to 3 heteroatoms, each of which may be optionally substituted by from 1 to 3 substituents independently selected from halo, C₁-C₆ alkyl, C₂-C₆ alkenyl, C₃-C₇ cycloalkyl, C₁-C₆ alkoxy, hydroxy, amino, cyano and C₁-C₃ perfluoro alkyl;
or R⁴³ and R⁴⁴ may be taken together to form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, oxo, hydroxy, amino, cyano and C₁-C₃ perfluoro alkyl;
Y is absent or selected from O, NH, and C₁-C₆ alkyl;
R⁴⁵ is selected from the group consisting of H, aryl, —(C₁-C₆ alkyl)-O—(C₁-C₆ alkyl), (C₁-C₆ alkyl)-NR⁴⁶R⁴⁷, —CO₂R⁴⁸, —O—(CH₂)₆—CO₂R⁴⁸, and —C(═O)NR⁴⁶R⁴⁷,
R⁴⁶ and R⁴⁷ independently selected from the group consisting of H, C₁-C₈ alkyl, C₂-C₈ alkenyl, C₂-C₈ alkynyl, —(C₁-C₆ alkyl)-O—(C₁-C₆ alkyl), aryl, aralkyl, heteroaryl, C₃-C₇ cycloalkyl, a three to twelve membered heterocyclic ring containing up to 3 heteroatoms, each of which may be optionally substituted by from 1 to 3 substituents independently selected from halo, C₁-C₆ alkyl, C₂-C₆ alkenyl, C₁-C₆ alkoxy, hydroxy, amino, cyano and C₁-C₃ perfluoro alkyl;
or R⁴⁶ and R⁴⁷ may be taken together to form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, oxo, hydroxy, amino, cyano and C₁-C₃ perfluoro alkyl;
R⁴⁸ is selected from the group consisting of H, aryl, aralkyl heteroaryl, C₁-C₆ alkyl, —(C₁-C₆ alkyl)-O—(C₁-C₆ alkyl), —(C₁-C₆ alkyl)-NR⁴⁶R⁴⁷, —(C₁-C₆ alkyl)-O—(C₁-C₆ alkyl)-O—(C₁-C₆ alkyl), each of which may be optionally substituted by from 1 to 3 substituents independently selected from halo, C₁-C₆ alkoxy, hydroxy, amino, cyano and C₁-C₃ perfluoroalkyl;
a is selected from 0 to 6;
b is selected from 0 to 6;
e is selected from 2 to 6;
R⁵ is selected from the group consisting of H, C₁-C₆ alkyl, —(CH₂)_{d}—C(═O)—NR⁵³R⁵⁴, —C(═O)—(CH₂)_{d}—NR⁵³R⁵⁴, and —C(═O)—X—R⁵⁵;
R⁵³ and R⁵⁴ are independently selected from the group consisting of H, C₁-C₆ alkyl, C₂-C₈ alkenyl, (C₂-C₈ alkynyl, —(C₁-C₆ alkyl)-O—(C₁-C₆ alkyl), —(C₁-C₆ alky)-NR⁵⁶R⁵⁷, —(C₁-C₆ alkyl)-C(═O)NR⁵⁶R⁵⁷, aryl, aralkyl, heteroaryl, C₃-C₇, cycloalkyl, a three to twelve membered heterocyclic ring containing up to 3 heteroatoms, each of which may be optionally substituted by from 1 to 3 substituents independently selected from halo, C₁-C₆ alkyl, C₁-C₆ alkenyl, C₃-C₇ cycloalkyl, C₁-C₆ alkoxy, hydroxy, amino, cyano and C₁-C₃ perfluoro alkyl;
or R⁵³ and R⁵⁴ may be taken together to form a three to twelve membered heterocyclic ring having tip to 3 heteroatoms which is optionally substituted by from 1 to 3 substituents independently selected from halo, C₁-C₆ alkyl, C₂-C₆ alkenyl, C₁-C₆ alkoxy, C₃-C₇ cycloalkyl, oxo, hydroxy, amino, cyano and C₁-C₃ perfluoro alkyl;
R⁵⁵ is selected from the group consisting of H, aryl, —(C₁-C₆ alkyl)-O—(C₁-C₆ alkyl), —(C₁-C₆ alkyl)-NR⁵⁶R⁵⁷, —CO₂R⁵⁸, —O—(CH₂)_{c}—CO₂R⁵⁸, and —C(═O)NR⁵⁶R⁵⁷,
R⁵⁶ and R⁵⁷ independently selected from the group consisting of H, C₁-C₈ alkyl, C₂-C₈ alkenyl, C₂-C₈ alkynyl, —(C₁-C₆ alkyl)-O—C₁-C₆ alkyl), aryl, aralkyl, heteroaryl, C₃-C₇ cycloalkyl, a three to twelve membered heterocyclic ring containing up to 3 heteroatoms, each of which may be optionally substituted by from 1 to 3 substituents independently selected from halo, C₁-C₆ alkyl, C₂-C₆ alkenyl, C₁-C₆ alkoxy, hydroxy, amino, cyano and C₁-C₆ perfluoro alkyl;
or R⁵⁶ and R⁵⁷ may be taken together to form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, oxo, hydroxy, amino, cyano and C₁-C₃ perfluoro alkyl;
R⁵⁸ is selected from the group consisting of H, aryl, aralkyl, heteroaryl, C₁-C₆ alkyl, —(C₁-C₆ alkyl)-O—(C₁-C₆ alkyl), —(C₁-C₆ alkyl)-NR⁵⁶R⁵⁷, —(C₁-C₆ alkyl)-O—(C₁-C₆ alkyl)-O—(C₁-C₆ alkyl), each of which may be optionally substituted by from 1 to 3 substituents independently selected from halo, C₁-C₆ alkoxy, hydroxy, amino, cyano and C₁-C₃ perfluoroalkyl;
d is selected from 0 to 6;
e is selected from 0 to 6;
R⁶ is selected from the group consisting of H, C₁-C₆ alkyl, —(CH₂)ᵣ—C(═O)—NR⁶³R⁶⁴, —C(═O)—(CH₂)ᵣ—NR⁶³R⁶⁴, and —C(═O)—X—R⁶⁵;
R⁶³ and R⁶⁴ are independently selected from the group consisting of H, C₁-C₈ alkyl, C₂-C₈ alkenyl, C₂-C₈ alkynyl, —(C₁-C₆ alkyl)-O—(C₁-C₆ alkyl), —(C₁-C₆ alkyl)-NR⁶⁶R⁶⁷, —(C₁-C₆ alkyl)-C(═O)NR⁶⁶R⁶⁷, aryl, aralkyl, heteroaryl, C₃-C₇ cycloalkyl, a three to twelve membered heterocyclic ring containing up to 3 heteroatoms, each of which may be optionally substituted by from 1 to 3 substituents independently selected from halo, C₁-C₆ alkyl, C₂-C₆ alkenyl, C₃-C₇ cycloalkyl, C₁-C₆ alkoxy, hydroxy, amino, cyano and C₁-C₃ perfluoro alkyl;
or R⁶³ and R⁶¹ may be taken together to form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, C₃-C₇ cycloalkyl, oxo, hydroxy, amino, cyano and C₁-C₃ perfluoro alkyl;
R⁶⁵ is selected from the group consisting of H, aryl, (C₁-C₆ alkyl)-O—(C₁-C₆ alkyl), —(C₁-C₆ alkyl)-NR⁶⁶R⁶⁷, —CO₂R, —O—(CH₂), —CO₂R⁶⁸, and —(═O)NR⁶⁶R⁶⁷,
R⁶⁶ and R⁶⁷ independently selected from the group consisting of H, C₁-C₅ alkyl, C₂-C₈ alkenyl, C₂-C₈ alkynyl, —(C₁-C₆ alkyl)-O—(C₁-C₆ alkyl), aryl, aralkyl, heteroaryl, C₃-C₇ cycloalkyl, a three to twelve membered heterocyclic ring containing up to 3 heteroatoms, each of which may be optionally substituted by from 1 to 3 substituents independently selected from halo, C₁-C₆ alkyl, C₂-C₆ alkenyl, C₁-C₆ alkoxy, hydroxy, amino, cyano and C₁-C₃ perfluoro alkyl;
or R⁶⁶ and R⁶⁷ may be taken together to form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, oxo, hydroxy, amino, cyano and C₁-C₃ perfluoro alkyl;
R⁶⁸ is selected from the group consisting of H, aryl, aralkyl, heteroaryl, C₁-C₆ alkyl, —(C₁-C₆ alkyl)-O—(C₁-C₆ alkyl), —(C₁-C₆ alkyl)-NR⁶⁶R⁶⁷, —(C₁-C₆ alkyl)-O—(C₁-C₆ alkyl)-O—(C₁-C₆ alkyl), each of which may be optionally substituted by from 1 to 3 substituents independently selected from halo, C₁-C₆ alkoxy, hydroxy, amino, cyano and C₁-C₃ perfluoroalkyl;
r is selected from 0 to 6;
s is selected from 0 to 6;
n is selected from 0 to 4;
m is selected from 0 to 3; and
p is selected from 0 and 1.

12. The method of claim 11, wherein the selective inhibitor of ROCK2 is a compound according to Formula XIV: 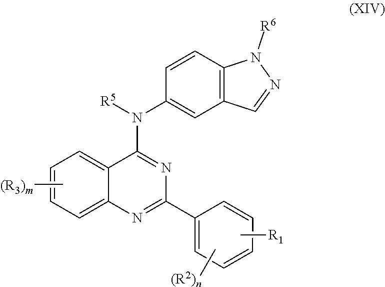
or a pharmaceutically acceptable salt, ester, stereoisomer, polymorph, solvate, N-oxide, or isotopically labeled compound thereof, wherein R¹, R², R³, R⁵, R⁶, m and n are as defined in Formula XIII.

13. The method of claim 12, wherein the selective inhibitor of ROCK2 is a compound according to Formula XV: 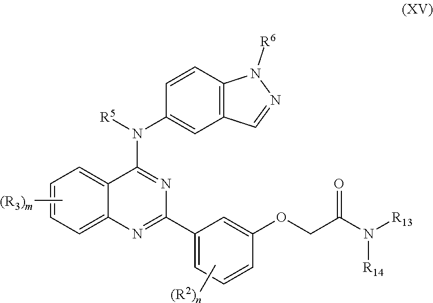
or a pharmaceutically acceptable salt, ester, stereoisomer, polymorph, solvate, N-oxide, or isotopically labeled compound thereof, wherein
R¹³ and R¹⁴ are independently selected from the group consisting of H, C₁-C₈ alkyl, C₂-C₈ alkenyl, C₂-C₈ alkynyl, —(C₁-C₆ alkyl)-O—(C₁-C₆ alkyl), —(C₁-C₆ alkyl)-NR¹⁶R¹⁷, —(C₁-C₆ alkyl)-C(═O)NR¹⁶R¹⁷, aryl, aralkyl, heteroaryl, C₃-C₇ cycloalkyl, a three to twelve membered heterocyclic ring containing tip to 3 heteroatoms, each of which may be optionally substituted by from 1 to 3 substituents independently selected from halo, C₁-C₆alkyl, C₂-C₆ alkenyl, C₃-C₇ cycloalkyl, C₁-C₆ alkoxy, hydroxy, amino, cyano and C₁-C₃ perfluoro alkyl;
or R¹³ and R¹⁴ may be taken together to form a three to twelve membered heterocyclic ring having up to 3 heteroatoms which is optionally substituted by from 1 to 3 substituents independently selected from halo, C₁-C₆ alkyl, C₂-C₆ alkenyl, C₁-C₆ alkoxy, C₃-C₇ cycloalkyl, oxo, hydroxy, amino, cyano and C₁-C₃ perfluoro alkyl;
R¹⁶ and R¹⁷ are independently selected from the group consisting of H, C₁-C₆ alkyl, C₂-C₈ alkenyl, C₂-C₈ alkynyl, —(C₁-C₆ alkyl)-O—(C₁-C₆ alkyl), aryl, aralkyl, heteroaryl, C₃-C₇ cycloalkyl, a three to twelve membered heterocyclic ring containing up to 3 heteroatoms, each of which may be optionally substituted by from 1 to 3 substituents independently selected from halo, C₁-C₆ alkyl, C₂-C₆ alkenyl, C₁-C₆ alkoxy, hydroxy, amino, cyano and C₁-C₃ perfluoro alkyl;
or R¹⁶ and R¹⁷ may be taken together to form a three to twelve membered heterocyclic ring having tip to 3 heteroatoms which is optionally substituted by from 1 to 3 substituents independently selected from halo, C₁-C₆ alkyl C₂-C₆ alkenyl, C₁-C₆ alkoxy, oxo, hydroxy, amino, cyano and C₁-C₃ perfluoro alkyl; and
wherein each of R², R³, R⁵, R⁶, m and n are as defined in Formula XIII.

14. The method of claim 13, wherein the selective inhibitor of ROCK2 is a compound according to Formula XVI: 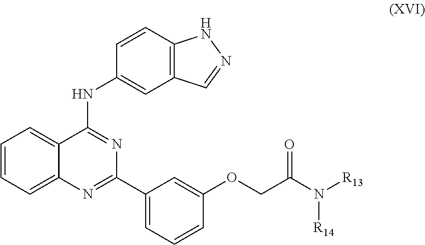
or a pharmaceutically acceptable salt, ester, stereoisomer, polymorph, solvate, N-oxide, or isotopically labeled compound thereof, wherein R¹³ and R¹⁴ are as defined in Formula XV.

15. The method of claim 14, wherein the selective inhibitor of ROCK2 is (2-(3-(4-((1H-indazol-5-yl)amino)quinazolin-2-yl)phenoxy)-N-isopropylacetamide) having the chemical Formula XVII: 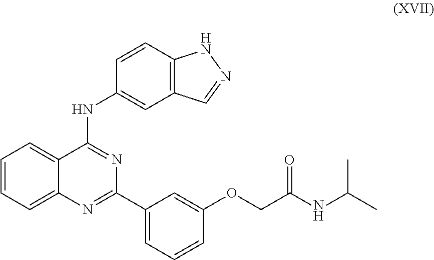
or a pharmaceutically acceptable salt, ester, stereoisomer, polymorph, solvate, N-oxide, or isotopically labeled compound thereof.

16. The method of claim 1, wherein the selective inhibitor of ROCK2 is a ribonucleic acid (RNA), optionally an antisense RNA against ROCK2 transcription, a small interfering RNA (siRNA), or a micro RNA (miRNA).

17. (canceled)

18. (canceled)

19. The method of claim 1, wherein the subject is a human.

20. The method of claim 1, wherein the subject has previous been administered an additional muscular dystrophy treatment, is concurrently being administered an additional muscular dystrophy treatment, or will subsequently be administered an additional muscular dystrophy treatment.

21. (canceled)

22. (canceled)

23. The method of claim 20, wherein the additional muscular dystrophy treatment is selected from the group consisting of a corticosteroid, an angiotensin-converting enzyme (ACE) inhibitor, a beta blocker, an exon skipping oligonucleotide, and a gene therapy.

24. The method of claim 23, wherein the corticosteroid is prednisone or deflazacort, the exon skipping oligonucleotide is eteplirsen or golodirsen, or the gene therapy is SRP-9001 or PF-06939926.

25. (canceled)

26. (canceled)
